(19) United States
(12) Patent Application Publication
(10) Pub. No.: US 2003/0044980 A1
(43) Pub. Date: Mar. 6, 2003

(54) Methods for low background cloning of DNA using long oligonucleotides

(75) Inventors: Ricardo Mancebo, San Bruno, CA (US); Kenneth B. Beckman, Alameda, CA (US); Sepp Saljoughi, Alameda, CA (US)

Correspondence Address: QUINE INTELLECTUAL PROPERTY LAW GROUP, P.C., P O BOX 458, ALAMEDA, CA, 94501, US

(73) Assignee: Gorilla Genomics, Inc.

(21) Appl. No.: 10/164,297

(22) Filed: Jun. 5, 2002

Related U.S. Application Data
(60) Provisional application No. 60/296,162, filed on Jun. 5, 2001.
(60) Provisional application No. 60/296,038, filed on Jun. 5, 2001.
(60) Provisional application No. 60/327,351, filed on Oct. 4, 2001.

(51) Int. Cl. C12N 15/85; C12P 19/34

(52) U.S. Cl. 435/455; 435/91.2; 435/320.1

(57) ABSTRACT

This invention provides methods for the assembly and cloning of target DNAs. Methods for cloning long chemically synthesized oligonucleotides without prior purification are provided. Compromised vectors are used to allow screening or selection for the desired target DNAs. Methods for assembling full-length target DNAs from smaller subsequences are provided, as are methods for purifying oligonucleotides.

### CROSS-REFERENCE TO RELATED APPLICATIONS

This application is a non-provisional utility patent application claiming priority to and benefit of the following provisional patent applications: U.S. S No. 60/296,162 filed Jun. 5, 2001, entitled “Methods for the Error Free Synthesis of DNA Molecules” by Beckman et al.; U.S. S No. 60/327,351, filed Oct. 4, 2001, entitled “Method for Cloning Long Oligonucleotides by Oligomer Priming on a Vector Template” by Mancebo et al.; and, U.S. S No. 60/296,038, filed Jun. 5, 2001, entitled “Methods for Very Low Background Cloning of DNA” by Mancebo et al. The present application claims priority to and benefit of each of these prior applications, each of which is incorporated by reference. The present application is also related to U.S. S No. 60/273,812, filed Mar. 6, 2001, entitled “A method for Purifying Full-length DNA Oligonucleotides Using Site-Specific Endonucleases,” by Mancebo et al., which is incorporated herein by reference.

### FIELD OF THE INVENTION

The present invention is in the field of nucleic acid synthesis and cloning. The invention includes methods for synthesis, assembly and cloning of target nucleic acids, including methods that incorporate the use of compromised vectors and long oligonucleotides. The invention also includes methods for purifying oligonucleotides and using nucleic acids in a variety of contexts.

### BACKGROUND OF THE INVENTION

The completion of the human genome project and the genome projects of other organisms has resulted in a need for access to physical reagents for use in functional genomics. Functional genomics involves, for example, the use of a full-length coding region of a gene in expression studies (e.g., over-expression studies), in which the full-length coding region is inserted into an expression vector and transformed into a host organism in such a way that the encoded protein's structure or function can be studied. High-throughput versions of such experimental approaches require access to validated libraries of tens of thousands of full-length clones, a resource that currently does not exist, due to limitations in methods for generating such clones. In addition to the study of proteins encoded by wild type coding sequences, future work in the growing field of proteomics will result in a need for large-scale alterations of genes, for purposes including expressing such genes and gene fragments in heterologous systems (for example, the expression of different variants of a human gene in a bacterial host).

One general approach to generating a protein-encoding DNA fragment is to clone such fragments directly as cDNA libraries, or to clone them from DNA fragments amplified from mRNA or cDNA by a method such as the polymerase chain reaction (PCR). There are significant limitations to currently existing methods. First, they rely on starting template material derived from mRNA. As a result, a gene has a significant probability of being absent in a given mRNA population because that gene may be unexpressed or of a low abundance in the source tissue. Also, mRNA is an unstable molecule that is prone to hydrolysis, which makes recovering longer fragments particularly difficult even when present in high concentrations. Second, the exact sequence represented in the source material is not determined until after the cDNA is fully sequenced, which means that it is impossible by cloning to specify the desired sequence before undertaking its manufacture. In fact, in the case of cloning cDNA libraries, the identity of any given clone is not known at all until some sequencing has been performed. Third, neither cloning nor amplification of cDNAs is a robust method suited to the high-throughput manufacturing of coding sequences. Fourth, the resulting products can include unwanted 5′ and 3′ flanking sequences (in the case of cloning) and/or mutations introduced by the process itself, the latter problem being particularly acute in the case of amplification. Fifth, these methods are not useful for delivering any variants of the native sequence, so they are of no use in optimizing protein-encoding regions for expression or for otherwise altering protein-encoding regions.

A second general approach to generating protein-encoding DNA fragments is their direct assembly from chemically synthesized single-stranded oligonucleotides, which has the following advantages. First, it does not rely on biological starting material, as the starting material is entirely chemical. Second, it permits the exact sequence to be specified in advance. Third, being a synthetic process, it is more amenable to robust manufacturing than constructing cDNA libraries, or amplifying from libraries. Fourth, the sequences delivered are free of undesired flanking regions. Fifth, because the specific sequence of each coding region is determined prior to manufacture, any protein sequence can be directly altered. Applications for altered coding sequences include protein mutagenesis for studies on protein function, protein re-encoding for uses such as expression in a heterologous system, scanning mutagenesis of important amino acid residues, site-directed mutagenesis of suspected binding regions, and so on.

Based on all of the reasons listed above, chemical synthesis of protein-encoding DNA fragments holds great promise as a tool for furthering functional genomics. The assembly of these fragments can be used to construct full-length synthetic genes of any desired sequence. Gene synthesis, however, is currently a limiting process characterized by high cost and low throughput. The high cost of gene synthesis is derived from three principal components: oligonucleotide costs, time and labor expenses involved in assembling oligonucleotides into larger fragments, and costs generated from sequencing multiple replicate clones to identify the positive clones.

The low throughput of gene synthesis results from problems inherent in assembling numerous oligonucleotides into a single fragment. For example, a gene of average length may involve the combination of dozens of oligonucleotides 50 bases in length in one reaction, providing an opportunity for the generation of thousands of undesired products due to non-specific hybridizations and ligations. Typically, the desired product is separated from undesired products by screening methods such as gel electrophoresis and size determination, or by sequencing, which affect the overall throughput of the process.

Increasing the length of the synthetic oligonucleotides reduces the problems inherent in assembly but introduces other difficulties. During DNA oligonucleotide synthesis, the number of active sites decreases as oligomer synthesis proceeds due to a coupling efficiency that is less than 100% for each base addition. This results in a reduction in the number of available active sites during each step in the synthesis reaction, leading to an overall reduction in the amount of product containing 5′ termini. Internal deletions within oligomers can also occur during the synthesis reactions due to deblocking and capping efficiencies that are below. 100% for each cycle. Therefore, as the length of an oligonucleotide increases, the yield of the full-length product at the end of a synthesis run decreases.

An example of the effect of oligonucleotide length on the yield of 5′ termini-containing product can be shown for an oligomer that is 20 bases in length, and an oligomer that is 100 bases in length. At 98% coupling efficiency, the predicted yield of a full-length 20-mer is (0.98)²⁰ or 66.8%, while the predicted yield of a full-length 100-mer is (0.98)¹⁰⁰ or 13.3%. As the length of synthesized oligonucleotides becomes greater, methods will be required to allow the specific isolation and recovery of small fractions of full-length oligomers from pools containing truncated oligos that include n-1, n-2, and larger deletion products.

A variety of well-established recombinant DNA methods have been developed for the cloning of DNA fragments. The majority of these involve the transformation of bacterial host cells with a recombinant product, molecules composed of a vector backbone (typically a double-stranded plasmid molecule engineered to accept the integration of additional DNA molecules) and a double-stranded target DNA insert, which is the material generally considered to be “cloned.” In most of these methods, a final step requires the identification of a “positive” clone, namely, a bacterial colony derived from the transformation of a bacterial cell by a single recombinant product, in which the hybrid DNA molecule contains the desired insert sequence. In contrast, a “negative” clone typically contains only the vector backbone itself (or some altered version of the backbone), and is often generated by the self-ligation of the vector or some fragment thereof. Another example of a negative clone is one which contains the vector backbone and an insert, but in which the insert contains a mutation such as a deletion, insertion, or point mutation. Because the cloning process generates a significant percentage of negative clones, multiple candidate clones are typically picked in order to ensure that at least one clone is positive. This screening step is time-, labor-, and resource-intensive, so in order to minimize the amount of work required in large-scale cloning, it is critical to minimize the “background” of negative clones. Ideally, a cloning method would be adequately efficient that a single colony could be picked with greater than 99% probability that it would be positive.

There are two standard methods for maximizing the percentage of positive clones. In the first, the relative concentration of vector and insert molecules combined in the “ligation” step is adjusted so that the probability of vector and insert recombining productively is maximal (typically, a vector:insert ratio of 1:3). In the second, the vector is modified (for example, through dephosphorylation) such that it can not self-ligate. These methods have disadvantages. For instance, adjusting the ratio of vector to insert requires that the vector and insert concentrations be determined, which itself requires that enough of these molecules be obtained in order to perform such determinations. Also, dephosphorylation of the vector decreases the overall efficiency of cloning by decreasing the efficiency of ligation. In any case, these methods typically result in a high enough percentage of negative clones that screening of multiple clones is nevertheless required in order to be assured of at least one positive clone.

Cloning background, e.g., the percentage of negative clones, becomes especially problematic when the amount of insert DNA is low. In such instances, for example, the need to optimize the ratio of vector to insert means that the overall amount of vector DNA must remain low, hence the overall number of transformants is correspondingly low. Any attempt to enhance the interaction between the vector and insert by increasing the overall amount of vector, and hence the vector:insert ratio, will likely result in obtaining fewer desired colonies due to self-ligation of vector molecules. Moreover, due to limitations of current technology, it is often difficult to measure very low concentrations of insert molecules, hence it is often unlikely to be able to optimize the ratio of vector to insert. As a consequence, the ratio of vector:insert can easily deviate very significantly from the ideal ratio and result in a large percentage of negative clones.

The present invention overcomes the above noted difficulties (e.g., the high cost, low throughput, and low efficiency of gene synthesis, and the low efficiency of cloning). A complete understanding of the invention will be obtained upon review of the following.

### SUMMARY OF THE INVENTION

The present invention provides several related strategies that provide for the efficient isolation and cloning of sequences of interest. The methods are particularly applicable to the isolation and/or cloning of chemically synthesized oligonucleotides (particularly large chemically synthesized oligonucleotides) without any need for oligonucleotide purification. Longer sequences assembled from synthetic oligonucleotides (e.g., full length genes, gene fragments, cDNA, or the like) are also a feature of the invention. In addition, generally applicable methods of oligonucleotide purification are provided. Compositions and kits which relate to each of the methods are also a feature of the invention.

Thus, in a first general class of methods, the invention provides megaprimer-mediated methods of cloning a target nucleic acid (typically, a target DNA) into a vector. In the methods, a first and second megaprimer and one or more nucleic acid that comprises or encodes the target DNA are provided. The one or more nucleic acid(s) include(s) at least one region of complementarity to or identity with the first megaprimer and at least one region of complementarity to or identity with the second megaprimer. The megaprimers are extended (typically via a polymerase mediated extension reaction) and the extended product is then intra molecularly ligated (e.g. with a ligase, e.g., in vitro) to form a functional vector. Optionally, the megaprimers are digested with one or more restriction enzymes to form ligation-compatible overlapping ends prior to the intramolecular ligation step.

The one or more nucleic acid(s) can consist of a single nucleic acid that at a first end comprises at least one region of complementarity to or identity with the first megaprimer and at a second end comprises at least one region of complementarity to or identity with the second megaprimer. Alternately, where the one or more nucleic acid includes at least two nucleic acids (and, optionally, more than two), an end of at least one of the at least two nucleic acids includes at least one region of complementarity to or identity with the first megaprimer and an end of at least one of the at least two nucleic acids includes at least one region of complementarity to or identity with the second megaprimer. If there are additional nucleic acids (more than two) in the overall set of nucleic acid(s) that encode the target DNA, then the set will typically include nucleic acids that are not complementary to the megaprimers, but, instead, are complementary to other members of the set.

The functional vector can be single or double-stranded. The megaprimers are typically single-stranded, but can be provided with their complementary strand. In one embodiment, the first and second megaprimers each comprise a nonfunctional marker or a fragment thereof, where the intramolecular ligation forms a functional marker (permitting selection of ligation products, e.g., by screening for the marker). The intramolecular ligation can be performed in vitro (e.g., using a ligase enzyme) or in vivo (e.g., by allowing a cell's endogenous ligase to perform the ligation). The marker can be any selectable marker, whether it confers an ability on a ligation product to replicate in a cell (e.g., by conferring antibiotic resistance, or by providing a functional origin of replication), or simply provides a property to be detected, whether in a cell or in vitro (e.g., in an in vitro transcription/translation system), such as a fluorescent, luminescent or fluorogenic protein (or nucleic acid that encodes such a protein). Common markers include genes/encoded proteins that confer cellular resistance to an antibiotic, resistance to ampicillin, resistance to tetracycline, resistance to kanamycin, resistance to neomycin, optically detectable markers (e.g., a marker nucleic acid that encodes a green fluorescent protein, or a marker nucleic acid that encodes a beta galactosidase protein), and/or the like. It will be appreciated that the marker can be a nucleic acid (gene), or a product encoded by the gene, depending on context. In any case, the method optionally includes transforming the vector into cells and selecting or screening the cells for expression of the marker.

In one typical embodiment, either the first or the second megaprimer comprises a nonfunctional marker or a fragment thereof and the one or more nucleic acid comprises a replacement sequence comprising a portion of the marker or its reverse complement. Integration of the replacement sequence with the nonfunctional marker results in generation (or regeneration) of a functional marker. The nonfunctional marker or replacement sequence can comprise one or more non-functional mutation of a functional marker, e.g., one or more deletion(s), insertion(s), and/or point mutation(s) (or fragment thereof) of the functional marker that renders the functional marker non-functional. The functional marker is formed/reformed upon integration (e.g., direct or indirect recombination) of the first and/or second megaprimer and the target nucleic acid. Here again, the functional marker resulting from integration of the megaprimer(s) and the target nucleic acid(s) can be any of those noted herein (e.g., vector components that provide for replication in a cell, resistance markers, optically detectable markers, or the like). Optionally, the target DNA comprises one or more additional open reading frame(s) or open reading frame subsequences. In one specific and useful embodiment, the target nucleic acid comprises an open reading frame located 5′ of and in frame with the replacement sequence. In this embodiment, expression of the functional marker provides an indication of the in frame expression of the target nucleic acid. In general, any products of this or any other method herein can be transformed into cells, which are selected or screened for expression of the marker resulting from integration.

The one or more nucleic acid can take any of a variety of forms. The cloning methods herein are particularly useful for the cloning of chemically synthesized oligonucleotides (particularly long oligonucleotides), as they can be cloned in the methods herein without purification, e.g., by selecting appropriate overlap properties with respect to, for example, the megaprimers. The one or more nucleic acids can include a single nucleic acid, e.g., where the nucleic acid is a single-stranded nucleic acid (e.g., typically, DNA) comprising or encoding the target nucleic acid/DNA, and having at least one region identical to a region of the first megaprimer 5′ of the target nucleic acid/DNA and at least one region complementary to the second megaprimer 3′ of the target nucleic acid/DNA. Alternately, the one or more nucleic acids can be a population of nucleic acids (e.g., overlapping nucleic acids) collectively having at least one region complementary or identical to a region of the first megaprimer 5′ of the target nucleic acid/DNA and at least one region complementary or identical to the second megaprimer 3′ of the target nucleic acid/DNA. The target nucleic acid can be provided in either single or double-stranded form.

Extension of the megaprimers can be carried out in a number of ways, including polymerase and ligase mediated methods. Most typically, polymerase-mediated methods are used, e.g., by annealing the single-stranded DNA to the second megaprimer, extending the second megaprimer, annealing the extended second megaprimer to the first megaprimer, and extending the first megaprimer and extended second megaprimer. This optionally includes denaturing the double-stranded product formed by extending the second megaprimer prior to annealing the extended second megaprimer to the first megaprimer (although this is not necessary—alternately a large excess of the appropriate components is added and the reaction is driven by mass action). In any case, the extension reactions can be done via standard polymerase extension reactions, or, conveniently, via PCR.

In a class of embodiments related to the foregoing embodiments, the invention includes methods of cloning a target DNA into a vector. In the methods, a first vector or vector template comprising a nonfunctional marker or fragment thereof is provided. One or more nucleic acid comprising or encoding the target DNA is also provided. The one or more nucleic acid has at least one region complementary to a strand of the first vector or vector template and a replacement sequence that includes a portion of the marker or its reverse complement. Integration of the replacement sequence with the nonfunctional marker results in a functional marker. The one or more nucleic acid is annealed to the first vector or vector template and extended. The resulting extended product is denatured and an extension primer capable of annealing to both 5′ and 3′ ends of the extended product is provided. The extension primer is annealed to the extended product and extended, forming a doubly extended product which is intramolecularly ligated to form a vector comprising a functional marker. The DNA polymerase used to extend the one or more nucleic acid or the extension primer optionally lacks strand displacement and/or 5′ to 3′ exonuclease activity. All of the above noted variations on the basic megaprimer cloning methods can be applied to this embodiment as well.

For example, as with the megaprimer cloning embodiments described above, the first vector or vector template can be a single-stranded vector, or can be a double-stranded vector, e.g., which can be denatured prior to annealing the one or more nucleic acid to the double-stranded vector. The one or more nucleic acid can consist of one nucleic acid, or can include at least two or more nucleic acids. The nonfunctional marker can include a mutation of a functional marker, e.g., deletion mutants, insertion mutants, point mutants, etc., as described above. The functional marker resulting from integration can be any of those noted herein or which are otherwise available, including, e.g., a selectable marker, a gene or encoded protein that confers cellular resistance to an antibiotic, a gene or encoded protein conferring resistance to ampicillin, a gene or encoded protein conferring resistance to tetracycline, a gene or encoded protein conferring resistance to kanamycin, a gene or encoded protein conferring resistance to neomycin, an optically detectable marker, a marker nucleic acid that encodes a green fluorescent protein, a marker nucleic acid that encodes a beta galactosidase protein, or the like.

Similar to the megaprimer cloning methods noted above, the ligation is typically performed in vitro, but can alternately be performed in vivo. In the case of in vitro ligation, the ligated doubly-extended product is introduced into cells which are selected or screened for expression of the marker. As above, the one or more nucleic acid is optionally a chemically synthesized oligonucleotide (or includes chemically synthesized oligonucleotides) that are at least 100 nucleotides, at least 150 nucleotides, at least 200 nucleotides, at least 250 nucleotides, or at least 300 or more nucleotides in length. Typically, the replacement sequence is proximal to the 5′ end of the oligonucleotide. The 5′ end of the oligonucleotide typically anneals before the 3′ end.

As in the methods above, the first vector or vector template optionally includes a second nonfunctional marker or fragment thereof and the one or more nucleic acid comprises a second replacement sequence that includes a portion of the second marker or its reverse complement, where integration of the second replacement sequence with the second nonfunctional marker results in a second functional marker. As in the megaprimer embodiments, any relationship of the first or second replacement sequence, with respect to each other or to the target DNA, can be used. For example, in one convenient embodiment, the target DNA includes an open reading frame located 5′ of and in frame with the second replacement sequence. The second functional marker can be any available marker, e.g., as noted herein.

As above, the method optionally includes transforming the doubly-extended product into cells and selecting or screening the cells for expression of the second marker resulting from integration of the second replacement sequence with the second non-functional marker.

In a related class of methods additional methods of cloning a target DNA into a vector are provided. In the methods, a linear first vector or vector template comprising a nonfunctional marker or fragment thereof is provided. One or more nucleic acid comprising or encoding the target DNA, the one or more nucleic acid comprising at least one region complementary to a strand of the first vector or vector template and a replacement sequence comprising a portion of the marker or its reverse complement, wherein integration of the replacement sequence with the nonfunctional marker results in a functional marker, is also provided. The one or more nucleic acid is annealed to the first vector or vector template, which is extended (e.g., using a polymerase). The resulting extended product is denatured and a primer comprising the reverse complement of the 3′ end of the extended product is provided. The primer is annealed to the extended product and extended (e.g., again, with a polymerase). The resulting doubly-extended product is intramolecularly ligated to form a functional vector comprising a functional marker.

All of the components and method steps can be varied essentially as noted above. For example, the linear first vector or vector template can be a linear double-stranded vector, which is denatured prior to annealing the one or more nucleic acid. The linear double-stranded vector is optionally produced by digestion with at least one restriction enzyme that cleaves a site located within the nonfunctional marker. The one or more nucleic acid can consist of one or of two or more nucleic acid(s). The nonfunctional marker can include a mutation of a functional marker, e.g., a deletion, an insertion, a point mutation and/or the like. The functional marker resulting from integration can include, e.g., a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, and/or a marker nucleic acid that encodes a beta galactosidase protein. The DNA polymerase used to extend the one or more nucleic acid or the primer optionally lacks strand displacement and/or 5′ to 3′ exonuclease activity. The ligation is optionally performed in vitro. The ligated doubly-extended product is optionally introduced into cells which are selected or screened for expression of the marker. Optionally, the one or more nucleic acid is a chemically synthesized oligonucleotide that is at least 100 nucleotides, at least 150 nucleotides, at least 200 nucleotides, at least 250 nucleotides, or at least 300 nucleotides or more in length. In this embodiment, the replacement sequence is optionally proximal to the 5′ end of the oligonucleotide. The linear first vector or vector template optionally includes a second nonfunctional marker or fragment thereof and the one or more nucleic acid optionally includes a second replacement sequence comprising a portion of the second marker or its reverse complement, wherein integration of the second replacement sequence with the second nonfunctional marker results in a second functional marker, which can be essentially any marker as noted herein. As above, the target DNA optionally includes an open reading frame located 5′ of and in frame with the second replacement sequence. Optionally, the doubly-extended product is transformed into cells which are selected and/or screened for expression of the second marker. The method optionally includes denaturing the one or more nucleic acid prior to annealing the one or more nucleic acid to the first vector or vector template. The doubly-extended product is optionally digested with at least one restriction enzyme prior to the intramolecular ligation. The first vector or vector template optionally comprises a functional selectable marker. These variations, and any others noted herein can be applied, as appropriate, to this embodiment.

In an additional class of related embodiments, the invention provides methods of cloning a target DNA into a vector. In the methods, a first vector or vector template comprising a nonfunctional marker or fragment thereof is provided. One or more nucleic acid that includes or encodes the target DNA is also provided, the one or more nucleic acid having at least one region complementary to a strand of the first vector or vector template and a replacement sequence that includes a portion of the marker or its reverse complement, where integration of the replacement sequence with the nonfunctional marker results in a functional marker. The one or more nucleic acid is annealed to the first vector or vector template. The one or more nucleic acid is extended on the template and the extended product is intramolecularly ligated to form a vector comprising a functional marker. Any of the above noted variations can be applied to this class of methods as well.

In an additional related class of embodiments, methods of cloning a target DNA into a vector are provided. In the methods, a first chemically synthesized oligonucleotide that is at least 100 nucleotides, at least 150 nucleotides, at least 200 nucleotides, at least 250 nucleotides, or at least 300 nucleotides in length is provided that comprises or encodes the target DNA, the first oligonucleotide comprising a first restriction site 5′ of the target and a region of sequence that is complementary to a first strand of the vector 3′ of the target. A second oligonucleotide primer with a second restriction site 5′ of a region of sequence complementary to a second strand of the vector and a first vector or vector template is also provided. At least one cycle of PCR amplification is performed to extend the provided oligonucleotides. The double-stranded product of the amplification is digested with a first restriction enzyme that cleaves the first restriction site and a second restriction enzyme that cleaves the second restriction site (for convenience, the first and second restriction enzymes can be the same, or can at least create ligation-compatible ends). The resulting product is intramolecularly ligated.

Any of the above noted variations can be applied to this method as well. In addition, in one aspect, the invention includes digesting with an enzyme that cleaves the provided first vector or vector template but not the product of the PCR amplification. An example useful restriction enzyme is Dpn I.

In an additional related class of methods, the invention provides methods of making a double-stranded DNA. In the methods, a plurality of oligonucleotides that are each at least 100 nucleotides (and more typically longer than 100 nucleotides, e.g., at least 150 nucleotides, at least 200 nucleotides, at least 250 nucleotides, or at least 300 nucleotides in length) and that collectively comprise a plurality of subsequences of the double stranded DNA are chemically synthesized. The plurality of oligonucleotides is assembled to form a plurality of genomers (these can be single or double stranded). The genomers are assembled to form the double-stranded DNA. At least one property of the double-stranded DNA (e.g., an activity of one or more encoded nucleic acid or polypeptide) is screened and/or selected for (e.g., by sequencing the DNA, restriction enzyme digestion of the DNA, or by cloning and expression of the DNA, or sequences associated with the DNA).

An advantage of the invention is that purification of oligonucleotides is not necessary to produce high-quality DNAs of interest. However, optionally, the methods include purifying the plurality of oligonucleotides, e.g., prior to assembly into genomers. For example, the oligonucleotides are optionally purified by enzymatic cleavage or by photocleavage. In addition, while the genomers do not need to be purified or quality checked prior to assembly into the DNA of interest, this step is also optionally performed. For example, at least one property of one or more of the genomers can be determined prior to assembling the genomers to form the double-stranded DNA. For example, optionally, the property of the genomer can be determined by sequencing the genomer, restriction enzyme digestion of the genomer, screening for expression of a marker fused to the genomer, or the like.

The present invention increases the efficiency of incorporation of inserts into vector backbone-containing molecules by any of a variety of strategies as noted above. In any of the methods, the invention optionally provides for the use of a large excess of vector to drive the efficient capture of an insert of interest. In general, the invention provides robust, high-throughput cloning of sequences of interest (including those encoded in chemically synthesized oligonucleotides) by optionally providing for the use of a single vector concentration and set of conditions for all cloning conditions, in the absence of any prior determination of insert concentration. The present invention also provides cloning methods that produce a low background of negative clones (e.g., those lacking any insert or those containing a mutated version of the desired target DNA). Similarly, the present invention also allows the direct cloning of long, chemically synthesized oligonucleotides without requiring a purification step. Another feature of the invention is an increase in the efficiency of assembly of subsequences to produce full-length target DNAs.

Although oligo purification is not generally required in the methods of the present invention, it can be performed, e.g., to increase the yield of cloned sequences that incorporate any oligonucleotides of interest. In addition, the present invention provides methods of purifying oligonucleotides, which can be applied to the methods herein, or which can be used as stand-alone purification methods. In the methods, a tagged target oligonucleotide is provided. The tagged target oligonucleotide includes the target oligonucleotide sequence and a tag 5′ of the target sequence. A bait oligonucleotide comprising a region complementary to the tag is also provided and the tagged target oligonucleotide and bait oligonucleotide are hybridized. The annealed oligonucleotides are digested with a nicking endonuclease that cleaves the tagged target oligonucleotide at a junction between the 3′ proximal end of the tag and the 5′ proximal end of the target (thereby releasing the target oligonucleotide).

In one useful class of embodiments, the nicking endonuclease-cleaves at a site that is 3′ of its recognition sequence, which can permit re-use of the bait oligonucleotide. Example nicking endonucleases with this activity are N.BstNBI and N.AlwI. The bait oligonucleotide typically includes a moiety for attaching the bait oligonucleotide to a solid support (biotin, an antibody ligand, or the like). The bait oligonucleotide is attached to the solid support before or after annealing the tagged target oligonucleotide and bait oligonucleotide.

The present invention also includes compositions, e.g., for practicing the methods herein, or which are produced by any of the methods herein. For example, the invention provides compositions comprising megaprimer pairs, e.g., the pair comprising a first megaprimer and a second megaprimer, where each megaprimer is a single-stranded DNA molecule that comprises a distinct portion of a vector backbone and a distinct portion of an essential marker (e.g., any sequence that is required for replication in a target cell, e.g., a sequence element required for replication of a plasmid, an origin of replication, a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, or the like).

In this class of embodiments, a first portion of the essential marker is typically proximal to the 5′ end of the first megaprimer and a second portion of the essential marker is typically proximal to the 5′ end of the second megaprimer. The vector backbone can include any typical backbone feature, e.g., an origin of replication, a selectable marker, a nonfunctional marker, an inducible promoter, a multiple cloning site, or the like. The composition can further comprise one or more chemically synthesized oligonucleotide that comprises, corresponds to, or encodes a target DNA.

In another aspect, the invention provides compositions that include a vector comprising at least one nonfunctional marker or fragment thereof, and one or more chemically synthesized oligonucleotide. The oligonucleotide is at least 100, at least 150, at least 200, at least 250, or at least 300 nucleotides in length, and includes at least one region complementary to at least one region of the vector, and a replacement sequence. The replacement sequence includes a portion of the marker or its reverse complement, where integration of the replacement sequence with the nonfunctional marker results in a functional marker.

In another aspect, the invention includes sets of synthetic oligonucleotides, e.g., where each synthetic oligonucleotide is at least 100 nucleotides, at least 150 nucleotides, at least 200 nucleotides, at least 250 nucleotides, or at least 300 nucleotides in length, wherein the oligonucleotides collectively comprise a genomer, gene, or other full-length DNA of interest. The set can include about 2 members, 5 members, 10 members, 20 members, 48 members, 96 members, 384 members, 1536 members, or more members. For example, the number of members can correspond to a standard sample handling system, e.g., comprising 96, 384, or 1536 well plates.

Kits provide an additional feature of the invention. For example, kits of the invention can include any of the compositions noted herein, e.g., with instructions for practicing the methods herein, containers for holding the compounds etc. of interest, packaging materials and/or the like.

### DEFINITIONS

An “essential marker” is a sequence element of a vector required either for the replication of the vector in a host cell or for the survival of a host cell under selected conditions, when transformed with the vector. Examples are a plasmid's origin of replication, an antibiotic resistance gene, or the like.

A “genomer” is a DNA molecule comprising a subsequence of a larger DNA of interest (e.g., a genomer could correspond to a portion of a gene), wherein the genomer is at least about 200 nucleotides (nt) (e.g., at least 300 nt, at least 400 nt, at least 500 nt, at least 600 nt, at least 700 nt, at least 800 nt) in length, and wherein one strand or portions of each strand were generated initially from synthetic oligonucleotides and thus, typically, comprise a predetermined sequence. A genomer can be single-stranded or double-stranded. Optionally, a genomer comprises a verified sequence, e.g., the genomer can be sequenced. A genomer can exist as an individual sequence or can be assembled into a larger nucleic acid of interest. A genomer can be cloned or uncloned. The genomer can include flanking sites.

A “megaprimer” is a single-stranded, double-stranded, or partially single-stranded DNA molecule that comprises a portion of one strand of a vector backbone. Megaprimers are generally supplied in pairs, where a pair of megaprimers (with their complementary strands in the case where the vector is double-stranded) comprise an entire functional vector backbone. If the vector is double-stranded, the megaprimers need not correspond to portions (e.g. halves) of the same strand of the vector backbone.

A “nicking endonuclease” is a site specific endonuclease that cleaves only one strand of the DNA on a double-stranded DNA substrate.

An “oligonucleotide” is a polymer of nucleotides or nucleotide analogues. The nucleotides can be natural or non-natural and can be unsubstituted, unmodified, substituted, or modified. A “long oligonucleotide” is a chemically synthesized oligonucleotide that is at least 100 nt in length, and which can be more than 100, e.g., 110, 120, 130, 150, 175, 200, 300 or more nt in length.

A “replacement sequence” is a nucleic acid segment whose integration with a nonfunctional marker (e.g., a mutated marker) results in a functional marker (e.g., a wild-type marker). A single-stranded replacement sequence can include either wild type or mutated marker sequences, and can correspond to either the coding strand or the non-coding strand of the marker.

A “synthetic oligonucleotide” is a chemically synthesized oligonucleotide, i.e., one made through in vitro chemical synthesis as opposed to one made either in vitro or in vivo by a template-directed, enzyme-dependent reaction.

A “vector backbone” is a nucleic acid comprising sequences necessary for the replication of the vector and its maintenance in a cell transformed with the vector. Examples include a plasmid's origin of replication. The backbone can further comprise elements added for convenience in subsequent cloning steps, such as a multiple cloning site, selectable marker, inducible promoter, etc. The backbone can be single-stranded or double-stranded.

### BRIEF DESCRIPTION OF THE FIGURES

FIG. 1, panels A-C, schematically depict a megaprimer-mediated cloning method.

FIG. 2, panels A-C, schematically depict an alternate megaprimer-mediated cloning method.

FIG. 3, Panels A-F schematically depicts the cloning of target sequences from either single-stranded or double stranded molecules by the specific priming and extension of target sequences on a denatured circular vector template.

FIG. 4, Panels A-E schematically depicts the cloning of target sequences from either single-stranded or double-stranded molecules by the specific priming and extension of target sequences on a denatured circular vector template, where the 5′ end of target sequence is first preferentially annealed to the vector.

FIG. 5, Panels A-E schematically illustrate the cloning of an oligonucleotide including the optional second replacement sequence.

FIG. 6, Panels A-F depicts the cloning of target sequences from either single-stranded or double stranded molecules by the specific priming and extension of target sequences on a denatured linear vector template.

FIG. 7, panels A-F schematically illustrate the cloning of target sequences from either single-stranded or double stranded molecules by the specific priming and extension of target sequences on a denatured linear vector template, where the nucleic acid comprising the target also comprises the optional second replacement sequence.

FIG. 8, Panels A-D schematically depict the use of a linear target sequence as the sole primer in a single extension reaction to clone target sequences by a heteroduplex-mediated method.

FIG. 9, Panels A-D schematically illustrate the cloning of an oligonucleotide including an optional second replacement sequence by a heteroduplex-mediated method.

FIG. 10, Panels A-C illustrate a method for cloning full-length long oligonucleotides using long oligomers as primers in PCR.

FIG. 11 is a flow chart schematically outlining three alternate gene assembly/analysis methods.

FIG. 12 schematically illustrates a method for purifying full-length oligonucleotides using photocleavage purification.

FIG. 13 schematically depicts the use of megaprimers to assemble genomers.

FIG. 14 schematically shows an oligonucleotide purification method in which a bait oligo is used to trap a tag on a target oligonucleotide.

FIG. 15 schematically depicts the megaprimer-mediated cloning of an oligonucleotide including the optional replacement sequence.

FIG. 16 schematically depicts genomer assembly by polymerase-mediated extension of oligonucleotides.

### DETAILED DESCRIPTION

Methods for synthesizing, assembling, and cloning target nucleic acids are provided, along with attendant compositions and kits. A target DNA can include any sequence(s) of interest, including but not limited to any gene, promoter sequence, coding sequence, exon sequence, intron sequence, untranslated sequence, and/or enhancer sequence.

Methods for cloning target DNAs are provided which use compromised vectors to reduce the background of negative clones. For example, the vectors are compromised by fragmenting the vector or by disrupting an essential marker on the vector. In the methods described herein, insertion of the target DNA into the compromised vector results in a functional vector competent for transforming, replicating inside, and/or optionally supporting the growth under selective conditions of host cells. The methods share the advantage that the background of negative clones derived from vector sequences has been minimized, which permits very low overall numbers of positive clones to be recovered efficiently. This advantage, in turn, permits cloning from very low amounts of insert material.

Optionally, the methods allow screening or selection against clones, e.g., where the target DNA contains an insertion or deletion. In these optional embodiments, the vector comprises a nonfunctional marker (e.g., a mutated or incomplete form of an antibiotic resistance gene or a mutated or incomplete form of a green fluorescent protein (GFP)). A nucleic acid insert is provided that comprises the target DNA and a replacement sequence. Integration of the replacement sequence supplied by the insert with the nonfunctional marker supplied by the vector results in a functional marker (e.g., a wild-type antibiotic resistance gene or a functional GFP). In preferred embodiments, the target DNA comprises an open reading frame (ORF) that is 5′ of and in frame with the replacement sequence, such that a fusion protein comprising the protein encoded by the ORF and the marker protein (e.g., GFP) is expressed. These embodiments allow screening or selection against undesired clones wherein the target DNA contains an insertion or deletion, since in many such clones the marker would no longer be in the correct reading frame.

The methods are particularly suited for cloning long unpurified synthetic oligonucleotides, since the methods are designed to favor cloning of full-length oligonucleotides over cloning of incomplete oligonucleotides lacking the 5′ end as a result of failed synthesis steps.

Another class of embodiments provides methods for assembly of genes (or other full-length double-stranded DNA targets of interest) from synthetic oligonucleotides. Additionally, the invention provides methods for purifying oligonucleotides. The following sections describe the invention in more detail.

Megaprimer Cloning

One aspect of the present invention provides new cloning strategies using megaprimers to clone nucleic acids of interest. These methods are particularly useful for the cloning of unpurified oligonucleotides (e.g., as the nucleic acid of interest), but are also generally applicable to the cloning of any single or double-stranded nucleic acid of interest. Indeed, the methods can be applied to the cloning of multiple target nucleic acids, e.g., genomers, e.g., to provide a full-length nucleic acid of interest. The megaprimers will often encode a non-functional fragment of a selectable marker that is rendered functional in a final clones; this strategy dramatically reduces background cloning of non-functional sequences. Further, this marker splitting approach can be applied to more than one component of the final clone, providing double or greater selection cloning schemes. For example, a megaprimer pair can encode a marker such as tetracycline split across the two megaprimers while simultaneously encoding a portion of a GFP protein for which the remaining portion is encoded as part of the nucleic acid of interest. One can then screen for tetracycline resistance and GFP production, providing for a double-selection of the final product clone. In general, either polymerase-mediated-assembly or ligation of clone components, or combinations thereof are used to assemble clones of interest. Any of these reactions can be performed in vitro or in vivo.

Thus, in one aspect, the invention includes methods of cloning a target DNA or other nucleic acid into a vector. In the methods, a first and second megaprimer (e.g., that each comprise a nonfunctional marker or a fragment thereof) are provided along with one or more nucleic acid that comprises or encodes the target DNA (e.g., a synthetic oligonucleotide) or other nucleic acid. The one or more nucleic acid includes at least one region of complementarity to or identity with the first megaprimer and at least one region of complementarity to or identity with the second megaprimer. The megaprimers are extended and the resulting product is intramolecularly ligated (typically in vitro, but optionally in vivo) to form a functional vector (which can be single or double-stranded and which typically includes a functional marker).

As noted, this method can be used to clone one or more nucleic acid of interest. For example, the one or more nucleic acid can consist of a single nucleic acid that at a first end comprises at least one region of complementarity to or identity with the first megaprimer and at a second end comprises at least one region of complementarity to or identity with the second megaprimer. Alternately, the one or more nucleic acid can comprise at least two nucleic acids, wherein an end of at least one of the at least two nucleic acids comprises at least one region of complementarity to or identity with the first megaprimer and an end of at least one of the at least two nucleic acids comprises at least one region of complementarity to or identity with the second megaprimer. In one embodiment, the one or more nucleic acid is a single-stranded DNA comprising or encoding the target DNA, in which the single-stranded DNA comprises at least one region identical to a region of the first megaprimer 5′ of the target DNA and at least one region complementary to the second megaprimer 3′ of the target DNA.

As noted, either the first or the second megaprimer optionally comprises a nonfunctional marker or a fragment thereof and the one or more nucleic acid comprises a replacement sequence comprising a portion of the marker or its reverse complement. Integration of the replacement sequence with the nonfunctional marker results in generation (or regeneration) of a functional marker. The nonfunctional marker or replacement sequence can comprise a non-functional mutation of a functional marker, e.g., a deletion, an insertion, and/or a point mutation (or fragment thereof) of the functional marker that renders the functional marker non-functional. The functional marker is formed/reformed upon integration (e.g., direct or indirect recombination) of the first and/or second megaprimer and the target nucleic acid. Here again, the functional marker resulting from integration of the megaprimer(s) and the target nucleic acid(s) can be any of those noted herein (e.g., vector components that provide for replication in a cell, resistance markers, optically detectable markers, or the like).

Optionally, the target DNA comprises one or more additional open reading frame(s) or open reading frame subsequences. For example, the target nucleic acid can comprise or encode an open reading frame subsequence that is part of the same open reading frame as the replacement sequence, e.g., where the functional marker is fused in frame to additional coding sequence encoded by target DNA (this can be useful when expression of the functional marker is used as an indicator of the reading frame of additional coding sequence). Alternately, the open reading frame can be a different open reading frame than the replacement sequence, or can be in frame with the replacement sequence open reading frame, but present as a separate open reading frame (e.g., where promoter or other elements are to be shared between the open reading frame that encodes the functional marker and the additional open reading frame), or where the formation of the functional marker is to be used as an indication of the reading frame of the target nucleic acid) or can be in a different reading frame. In one specific and useful embodiment, the target nucleic acid comprises an open reading frame located 5′ of and in frame with the replacement sequence. In this embodiment, expression of the functional marker provides an indication of the in frame expression of the target nucleic acid.

The marker(s) can include any known marker, e.g., a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, and/or a marker nucleic acid that encodes a beta galactosidase protein. A variety of markers are known in the art, e.g., as set forth in Berger, Sambrook, and Ausubel, infra. The megaprimers can be combined with each other to form a functional marker, or the megaprimers can be combined with the target nucleic acid to form a functional marker, or both. For example, in one embodiment, either the first or the second megaprimer comprises a nonfunctional marker or a fragment thereof and the one or more nucleic acid to be joined with the megaprimers comprises a replacement sequence comprising a portion of the marker or its reverse complement, wherein integration of the replacement sequence with the nonfunctional marker results in a functional marker. The nonfunctional marker can be rendered non-functional by any of a variety of strategies, including mutation of a functional marker by deletion, insertion, point mutation, or the like. Most typically, the vector is transformed into cells which are screened for expression of the marker. However, other strategies, such as expression in an in vitro transcription-translation system can also be used for selection of a marker, particularly where the marker is an optically selectable marker such as a luminescent or fluorescent marker (GFP, luciferase, or the like).

The megaprimers can be extended by any of a variety of strategies with respect to each other and the target nucleic acid. For example, in one embodiment, the method includes annealing a single-stranded DNA to the second megaprimer, extending the second megaprimer, annealing the extended second megaprimer to the first megaprimer, and extending the first megaprimer and extended second megaprimer. Optionally, the double-stranded product formed by extending the second megaprimer can be denatured prior to annealing the extended second megaprimer to the first megaprimer. Similarly, the intramolecular ligation of product nucleic acids can be performed by any available ligation method, including blunt-end ligation, or sticky end ligation (e.g., including digesting ends to be ligated with at least one restriction enzyme prior to the intramolecular ligation step), performed in vitro (e.g., using a ligase enzyme or chemical ligation strategy) or in vivo (e.g., allowing a cell to perform the ligation with the typical cellular repair machinery).

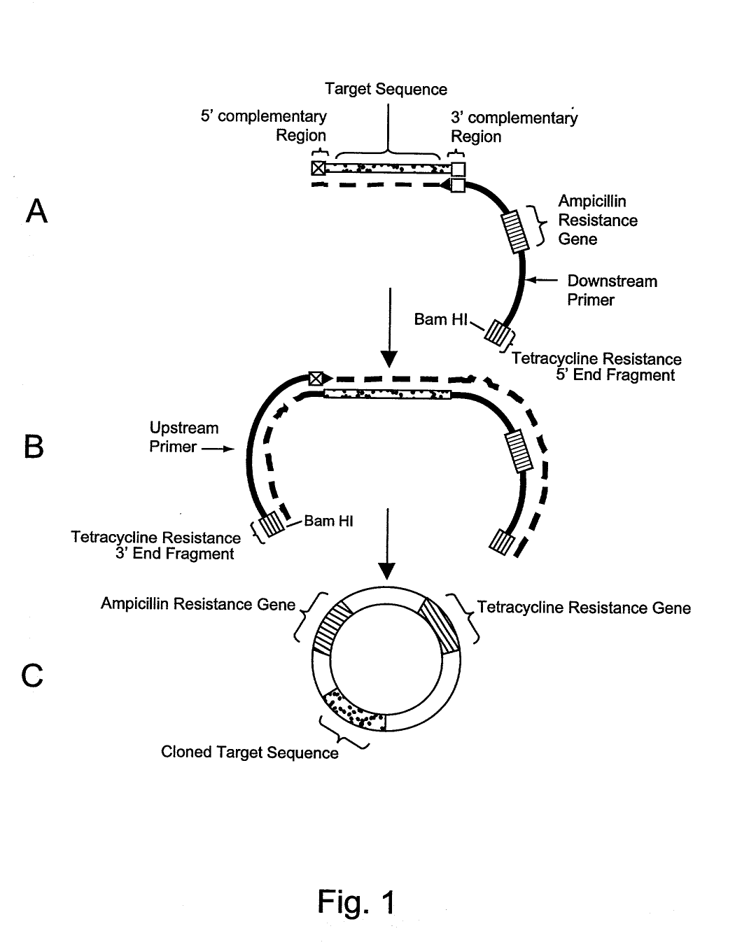

A first megaprimer cloning embodiment is illustrated in FIG. 1, Panels A-C. In brief overview, in panel A, the downstream megaprimer is annealed to a single stranded target sequence at a complementary region. The megaprimer is extended with a polymerase. In panel B, denaturation is followed by annealing of the upstream megaprimer to the extended target sequence at a complementary region and extension from both megaprimers. In panel C, the products of panel B are digested, ligated and transformed into E. coli and selected for tetracycline resistance.

In the embodiment of FIG. 1, a single-stranded target insert sequence, for example, a synthetic oligonucleotide, is converted into a circular vector-containing molecule using a megaprimer-mediated cloning strategy. Megaprimers are long, single-stranded DNA molecules which provide portions of a cloning vector backbone. In the embodiment depicted, each megaprimer provides one functional half of a vector backbone. In the final recombined molecule, the insert is flanked by these two megaprimer sequences, which are referred to here as the “upstream” and “downstream” megaprimers. The single-stranded target insert molecule is designed such that it has a sequence at its 5′ terminus that is identical to the 3′ end of the upstream megaprimer, and a sequence at its 3′ terminus that is the reverse complement of the 3′ end of the downstream megaprimer. These sequences are used in two cycles of intermolecular annealing and strand extension to convert the megaprimers and insert sequence into a single double-stranded linear sequence.

The reactions for doing so may be carried out in a single reaction chamber containing all three DNA molecules, or can be performed by first reacting the insert and downstream megaprimer and subsequently adding the upstream megaprimer. The reaction mixture also typically includes reagents known to those skilled in the art of in vitro synthesis of DNA, such as buffers, salts, deoxynucleotide triphosphates, and a DNA polymerase such as the Klenow fragment of E. coli DNA polymerase, or a thermostable polymerase such as that from Thennophilus aquaticus. In Step 1 (Panel A) of the embodiment depicted in FIG. 1, the single-stranded insert molecule and downstream megaprimer are allowed to anneal at their 3′ ends by controlling the temperature of the reaction, and then the 3′ ends of both molecules are extended by in vitro enzymatic DNA synthesis. The result of this extension is that the extended 3′ end of the downstream megaprimer is converted into the reverse complement of the 3′ end of the upstream megaprimer. Next, in Step 2 (Panel B), the 3′ ends of the upstream megaprimer and the extended downstream megaprimer are annealed and extended by in vitro DNA synthesis (polymerase mediated extension), as illustrated.

The annealing of these two megaprimers can be achieved either by denaturation and reannealing, for example through the heating and cooling of the solution, or can be achieved merely by using a large excess of upstream megaprimer as compared to the insert oligonucleotide, such that the breathing of the double-stranded insert-downstream megaprimer molecule permits strand invasion by the 3′ end of the upstream megaprimer to form a complex capable of extension. The result of these reactions is a double stranded molecule whose contiguous sequence is that of the upstream megaprimer, insert sequence, and downstream megaprimer, combined through their complementary regions.

In order to convert this linear double-stranded DNA molecule into a circularized vector for efficient cloning, the termini are ligated, e.g., as shown in Panel C of FIG. 1. This ligation can be achieved via blunt-end cloning, but is more preferably achieved by so-called “sticky-end” cloning, in which restriction digestion of the two ends generates compatible single-stranded overhangs that cause sequence-specific annealing and efficient recircularization, as the overlap increases the efficiency of the ligation reaction.

In order to achieve the highest possible number of transformants from a recircularization, it is useful to prevent concatomerization of the linear double-stranded molecules, since such reactions result in repetitive molecules that do not support transformation of bacterial cells, and since concatomerization decreases the abundance of single recircularized molecules. Diluting the reaction significantly can minimize concatomerization, such that the probability of intermolecular collision is minimized. In addition, the ligation reaction conditions may be biased in favor of intramolecular events, by (for example) using low concentrations of ligase and relatively higher temperatures, both of which will disfavor the inefficient intermolecular ligation, but which will have less effect on more efficient intramolecular recircularization reactions.

Finally, the recircularized vector is transformed into bacterial cells by methods known to those skilled in the art, and recombinant clones are selected on an appropriate growth medium. As shown in FIG. 1, an important element of the strategy of the depicted embodiment is the fact that the sequences of the 5′ ends of the megaprimers, and hence the termini of the double-stranded molecule to be recircularized, define two functional halves of an essential marker, defined as a sequence element which is required either for the replication of the plasmid or for the survival of the transformed host cell. Two examples of such essential markers are the origin of replication and/or an antibiotic resistance gene. In FIG. 1, selection by tetracycline resistance is illustrated, for the purpose of illustration. The selectable marker can be any biological marker known by those skilled in the art. The functional significance of such a design is that in the absence of both halves of the essential region, a viable transformant under the relevant selection conditions does not occur. This strategy minimizes the background of negative clones, since neither the megaprimers themselves, nor the insert-downstream primer double-stranded intermediate, is capable of supporting transformation. Only when the two megaprimers are converted into double stranded molecules through the interposition of the insert sequence, and the essential marker is restored through recircularization, will a viable plasmid be restored. Moreover, if the essential marker is such that any alteration of its sequence results in its functional disruption (for example, in the case of an antibiotic resistance gene), then this selection will also ensure that spurious recircularization is prevented, such as might occur due to ligation of damaged or otherwise incomplete molecules.

Long Oligos

One particularly useful aspect of the above embodiment is its application to the cloning of unpurified long oligonucleotides, which are characterized by a predominance of “failure sequences” prematurely terminated at their 5′ ends. Depending upon the length of the oligonucleotide, these failure sequences can represent the majority of the total population, which results in a need to purify the full-length oligos before they are used in further application, since such failure sequences typically interfere with the manipulation of the full-length oligo, and because the preponderance of failure sequences in a mixture make it difficult to quantify the amount of the minority full-length product. In the present invention, such failure sequences will not need to be removed. Although these failure sequences will anneal to the downstream megaprimer and be extended to yield a double-stranded intermediate, the resulting double-stranded molecules generally do not have adequate sequence complementary to the upstream megaprimer to be further extended.

In short, the use of a large excess of megaprimer arms, combined with the fact that these arms can only be converted into a viable plasmid by the interposition of a full-length or near full-length insert molecule, means that the background of spurious negative colonies will be low. This, in turn, permits the procedure to be carried out without prior quantification of the insert molecule, since the molar excess of megaprimer drives the consumption of the target insert sequences in the joining reactions by mass action, and “captures” the lower concentrations of insert by specific annealing and extension. This is especially useful when insert concentrations are very low.

Single-Stranded Nucleic Acid Comprising a Replacement Sequence

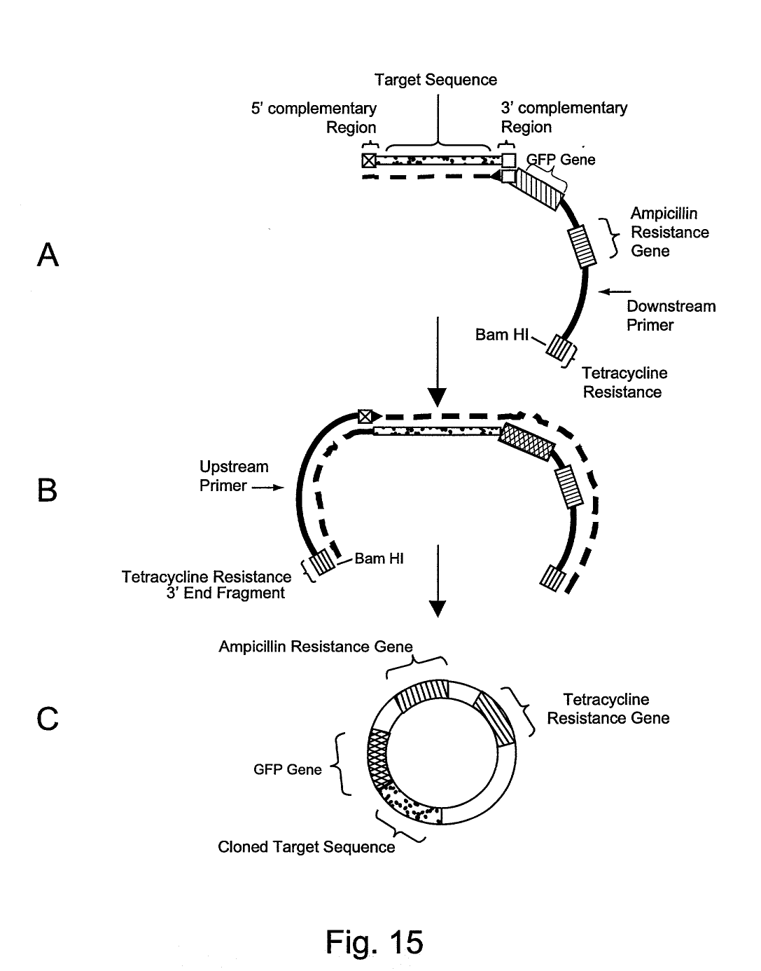

FIG. 15 illustrates the cloning of a single-stranded insert, for example a synthetic oligonucleotide, by the megaprimer method. In this example, the 3′ complementary region of the single-stranded nucleic acid insert comprises a replacement sequence for the nonfunctional GFP marker (reverse crosshatching) located on the downstream megaprimer, as shown in panel A.

The insert is cloned as in the embodiment described above. Briefly, the 3′ ends of the single-stranded insert and downstream megaprimer are annealed and then extended by in vitro enzymatic DNA synthesis. Optionally, the product is denatured. Next, the 3′ ends of the upstream megaprimer and the extended downstream megaprimer are annealed and extended by in vitro enzymatic DNA synthesis, as shown in panel B. The resulting double stranded product is ligated, either by blunt-end cloning or preferably by sticky-end cloning following restriction digestion. As above, ligation reforms a selectable marker that had been split between the megaprimers (in this example, the tetracycline resistance gene).

This method allows for the screening of insertion and deletion mutations in protein encoding target sequences by making fusions in-frame to the GFP gene (crosshatched) and then selecting the GFP positive colonies prior to sequencing.

Double-Stranded Target

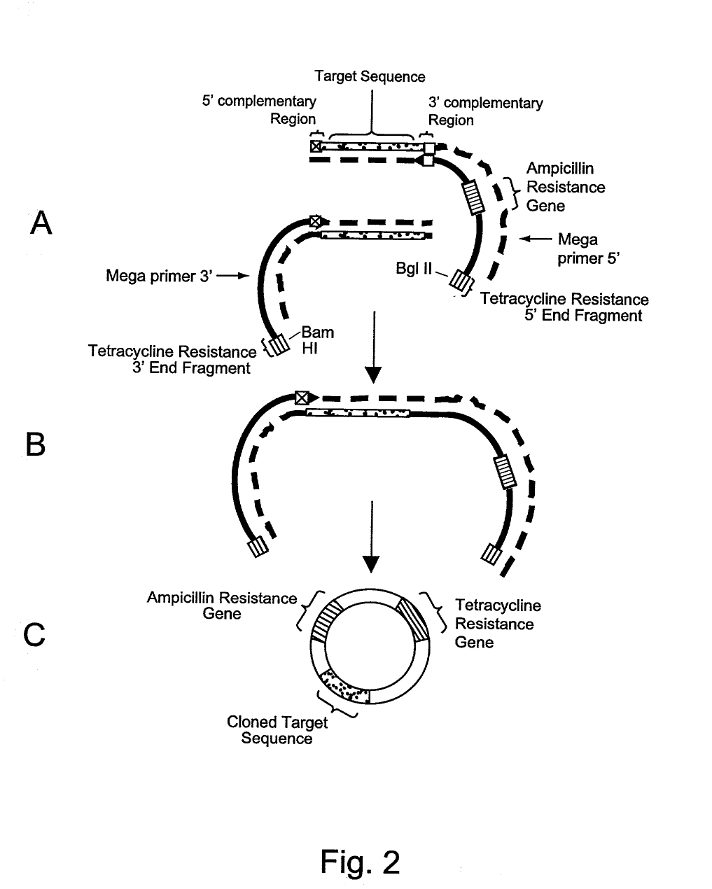

A second megaprimer cloning embodiment is illustrated in FIG. 2, Panels A-C. showing an example in which the insert sequence is double stranded. Briefly, the double stranded target sequence is denatured and the megaprimers are annealed 5′ and 3′ to the single-stranded target sequences at the complementary regions and extended (panel A). The 5′ and 3′ complementary extensions are denatured and annealed to target sequences and extension is performed from both megaprimers (panel B). The products (panel C) are digested, ligated and transformed into E. coli and selected for tetracycline resistance.

The embodiment of FIG. 2 is similar to the embodiment illustrated in FIG. 1, except for the fact that the insert sequence is a double-stranded molecule. As a result, the insert anneals to the complementary regions at the 3′ ends of both the upstream and downstream megaprimers, and can be extended to produce the intermediate double stranded molecules depicted in step 1 (Panel A). In step 2 (Panel B), the extended products are denatured, annealed, extended, and converted into a linear double-stranded molecule that contains the insert and both vector arms capable of recircularization into a full plasmid vector.

One of the potential problems with this embodiment is the potential for direct illegitimate mispriming between the two megaprimers via sequences of imperfect complementarity, or via their 3′ nucleotides, as is known to occur in (for instance) the formation of so-called “primer-dimers” in the polymerase chain reaction. The result of such an event is a double-stranded molecule with termini capable of recircularization, but which lacks the insert. Most such falsely primed molecules, since they will contain internal mismatches, fail to result in transformed colonies, owing to the tendency of DNA containing such mismatches not to survive transformation. Nevertheless, some such transformants can persist, and will represent a background of negative clones. Hence, it is a further aspect of the present invention that the megaprimer sequences may be specifically designed to prevent such inter-megaprimer mispriming. This may be achieved by an iterative process, in which clones resulting from reactions carried out in the absence of insert sequences (which are therefore a result of mispriming) are isolated and sequenced. Subsequent analysis of mispriming hotspots can be used to identify sequences responsible for mispriming, and these can be removed by traditional methods such as site-directed mutagenesis, resulting in a plasmid with a lower tendency for such mispriming.

Making Mega Primers

As noted herein, a “megaprimer” is typically a single-stranded DNA molecule that comprises a portion of one strand of a vector backbone. However, the megaprimers can be supplied with their complementary strand, if desired. Megaprimers are generally supplied in pairs (or as sets of more than 2 components that, when combined with a target nucleic acid provide a functional vector backbone), where a pair of megaprimers (optionally with their complementary strands) comprise an entire functional vector backbone. If the vector is double-stranded, the megaprimers need not correspond to portions (e.g. halves) of the same strand of the vector backbone. Single-stranded megaprimers can be made by a number of methods know to those skilled in the art, such as (for example) their generation as double-stranded products by restriction digestion from a parent molecule or their amplification from a template molecule, followed by their conversion to single-stranded molecules by any of a number of established methods. For example, one can perform asymmetric PCR to selectively produce desired single strands. Alternately, one can perform PCR amplification with selectively phosphorylated oligonucleotides followed by selective degradation of strands comprising a 5′ phosphate. For example, lambda exonuclease selectively degrades a first strand of a double-stranded molecule where the first strand comprises a 5′ phosphate, and leaves the second strand intact where the second strand does not comprise a 5′ phosphate group. The resulting single-stranded nucleic acid can subsequently be phosphorylated using standard techniques (e.g., treatment with a kinase enzyme), e.g., to facilitate subsequent ligation.

Insert A

One class of embodiments (referred to here as the “insert A” embodiments) allows the incorporation of a target sequence into a cloning vector by using one or more single-stranded or double-stranded nucleic acids comprising or encoding the target (e.g., a single-stranded nucleic acid insert can correspond to either strand of a double-stranded DNA target) as a primer. In this method, a nonfunctional marker located on the vector is converted to a functional marker by sequences supplied by the one or more nucleic acids that comprise the target. Selection or screening for the functional marker reduces the background of negative clones.

The vector used in the method may be single-stranded or double-stranded. The vector comprises a nonfunctional marker or a nonfunctional portion of a marker (e.g., a mutated or truncated antibiotic resistance gene). The nucleic acid insert comprises the target DNA, at least one region that is complementary to a strand of the vector (or optionally to a vector template strand, in the case of a single-stranded vector), and a replacement sequence. This replacement sequence comprises a portion of a functional version of the marker, such that integration of the replacement sequence supplied by the insert with the nonfunctional marker supplied by the vector or vector template results in a functional marker. The insert may comprise one or more single-stranded or double-stranded nucleic acids which singly or collectively comprise the target, region of complementarity to the vector or vector template, and replacement sequence.

In this method, the vector or vector template and insert are annealed. (Optionally, if the vector and/or insert is double-stranded, it may be denatured prior to the annealing step.) The nucleic acid insert is extended, preferably with an enzyme lacking strand displacement and/or 5′ to 3′ exonuclease activity (e.g., T4 DNA polymerase). The resulting product is denatured, and an extension primer that anneals to both the 5′ and 3′ end of the extended product is used in a second extension step (again, preferably with an enzyme lacking strand displacement and/or 5′ to 3′ exonuclease activity). Intramolecular ligation of the resulting doubly-extended product results in a circularized vector comprising a functional marker.

The ligation can be performed in vitro, followed by transformation of cells with the circularized vector. Alternatively, ligation can occur in vivo following transformation of the doubly-extended product into cells. Either method permits screening or selection of the resulting transformed cells for cells expressing the functional marker, which cells are likely to contain a vector carrying the desired target.

In one embodiment, the nucleic acid insert is a long synthetic oligonucleotide (e.g., an oligonucleotide that is at least 100 nt, at least 150 nt, at least 200 nt, at least 250 nt, or at least 300 nt in length). Optionally, the replacement sequence is located at or near the 5′ end of the oligonucleotide. Optionally, the conditions under which the oligonucleotide is annealed to the vector are controlled such that the 5′ end of the oligonucleotide anneals before the 3′ end. These options can obviate the need to purify the full-length oligonucleotide away from shorter oligonucleotides which lack 5′ ends as a result of failed synthesis steps prior to cloning the oligonucleotide.

Optionally, the vector or vector template can comprise a second nonfunctional marker or nonfunctional portion of a marker and the nucleic acid comprising the target can comprise a second replacement sequence, such that integration of the second replacement sequence and second nonfunctional marker results in a second functional marker. In one embodiment, the target DNA comprises an open reading frame located 5′ of and in frame with the second replacement sequence, such that a fusion protein comprising the protein or peptide encoded by the open reading frame and the marker protein is expressed. This embodiment permits selection or screening of transformed cells to select or screen against some undesired clones wherein the target DNA contains insertion(s), deletion(s), point mutation(s) or the like that disrupt the reading frame or expression of the marker.

The first and optional second marker can be any known to those of skill in the art, including but not limited to a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein. A nonfunctional version of such a marker may result from an insertion, deletion, or point mutation, for example.

In all embodiments, the vector can optionally comprise an additional, functional marker for use in propagating the vector.

### EXAMPLE

### Insert A Cloning of Single-Stranded or Double-Stranded Nucleic Acid

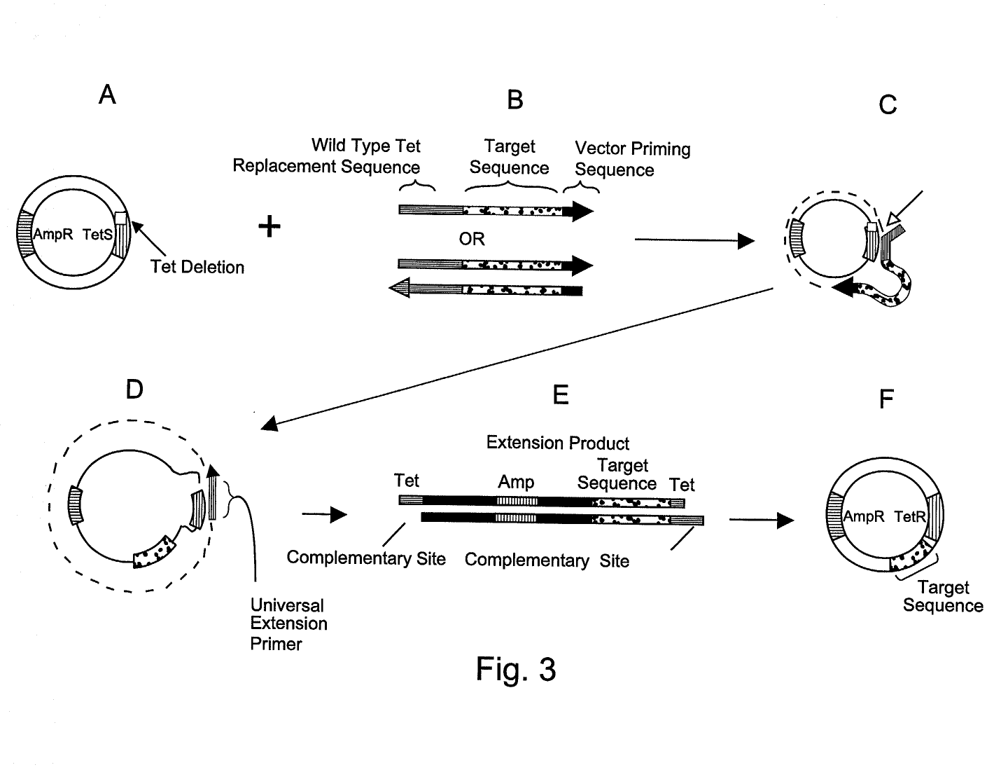

FIG. 3, panels A-F depicts the cloning of target sequences from either single-stranded or double stranded molecules by the specific priming and extension of target sequences on a denatured circular vector template. In brief overview, the double stranded vector of panel A is denatured. The resulting single stranded vector is annealed to a single or double stranded target sequence (panel B) and extended with T4 DNA polymerase (panel C). The resulting extension products are denatured, annealed with a universal extension primer and extended with polymerase (panel D). The extension primer anneals at the junction of the 5′ end of the tet gene and vector sequence and allows the extension of the second strand to generate a fully complementary extension product (panel E). The resulting product (panel F) is ligated, transformed into E. coli and selected with tetracycline.

In the method depicted in FIG. 3, a mutation in a selectable marker is converted to wild type by sequences supplied by the target primers. As depicted in panel A, a vector can include, for example, two selectable markers: one marker to propagate the vector (vertical hatching), and one mutated marker to select for clones containing the target insert. In this example, the ampicillin resistance gene (ampR) is the marker used to propagate the vector, and is shown here solely for the purpose of illustration, as any selectable marker know by those skilled in the art might be employed. The conversion of a deletion mutation in the tetracycline resistance gene (blank box by horizontal hatching) (TetS) into a wild type gene (horizontal hatching) (TetR) with sequences supplied by the insert (horizontal hatching on arrow) is used in this example as an assay to select the clones containing the target sequences (dotted). Any selectable marker known by those skilled in the art can also be employed in this assay.

Panel B illustrates that any single-stranded or double stranded target DNA molecule containing a region of complementarity to a vector template, referred to here as the vector priming sequence (solid filled), and replacement sequences for the mutated selectable marker can potentially be used as a primer in a cloning reaction. Panel C depicts an extension reaction of an annealed target primer with T4 DNA polymerase, where the extension reaction terminates at the first base on the vector template that is annealed to the primer (open arrow), owing to lack of strand displacement by this polymerase. Upon denaturation, this results in the generation of a single-stranded molecule containing sequences for the TetR gene at the 5′ end and sequences immediately upstream of the TetR gene at the 3′ end. T4 DNA polymerase is used in this example for the purpose of illustration, as any DNA polymerase that lacks a strand displacement activity such as Taq polymerase can be used in the extension reaction.

Panel D illustrates the conversion of the single-stranded molecule in Panel C to a double stranded complementary molecule with complementary overhangs. This involves denaturing the first extension product and annealing an extension primer that anneals to sequences at both the 5′ and 3′ ends of the extension product. This results in bridging the ends of the product template for a second round of extension by T4 DNA polymerase to generate a fully complementary extension product containing the target sequence, the AmpR gene, and a fragmented TetR gene, as shown in Panel E. Panel F depicts the final ligated product that results from the ligation, transformation, and selection with tetracycline of positive colonies.

This method of cloning has the advantage of being able to incorporate any linear DNA sequence into a cloning vector by using the target sequence as a primer, and then joining the vector in an intramolecular reaction without the need to digest prior to circularization. This means that any selectable marker mutation can be converted to a wild type sequence without having to rely on the natural restriction sites within the marker genes.

### Example

### Insert A Cloning with Selective Annealing of Oligonucleotide

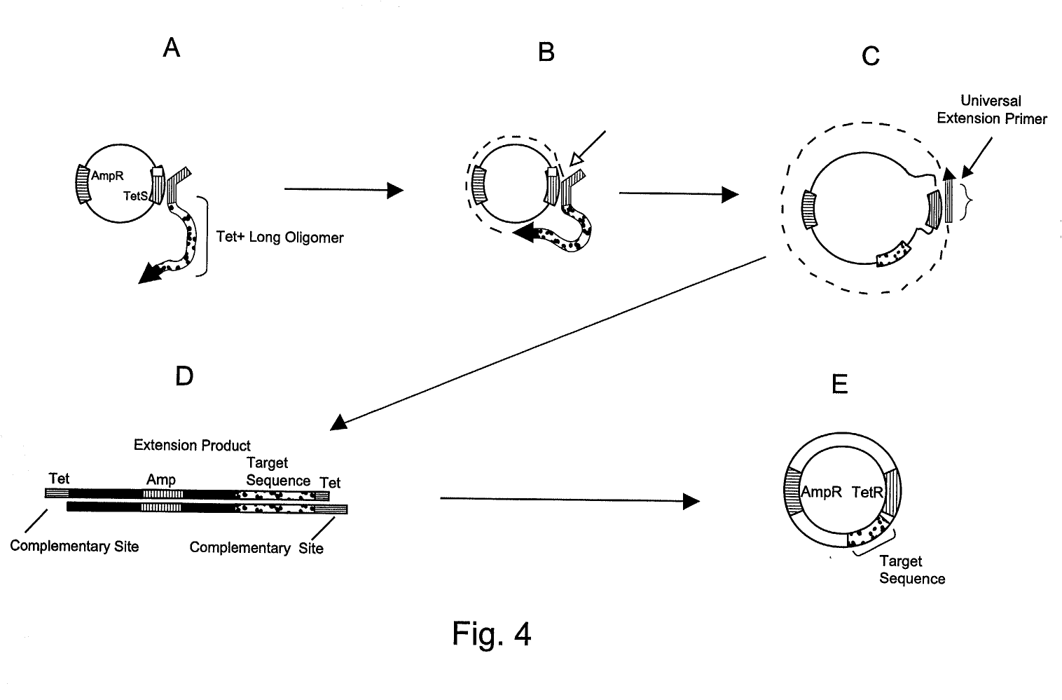

FIG. 4, Panels A-E depicts the cloning of an oligonucleotide by the insert A method. As DNA oligonucleotide synthesis proceeds, the number of active sites decreases due to a coupling efficiency that is less than 100% for each base addition. A reduction in the number of available active sites during each step in the synthesis reaction results in an overall reduction in the amount of full-length product that is synthesized. Therefore, as the length of an oligonucleotide increases, the yield of the full-length product at the end of a synthesis run decreases. As the need for the synthesis of longer oligonucleotides becomes greater, methods will be required that allow the specific isolation and recovery of small fractions of full-length oligomers from pools containing truncated oligomers. The advantage of the method described in this example is that oligomers containing 5′ ends can be selectively cloned from a mixed population of truncated oligomers without the need for purification.

As depicted in panel A, an oligonucleotide containing a 5′ phosphate is annealed first at the 5′ end to a vector template. Because the 5′ end has a higher melting temperature than the 3′ end, oligomers that contain 5′ end sequences are selectively annealed to the vector first, and oligomers that lack 5′ end sequences are excluded from annealing at higher temperatures. As in the previous example, the oligonucleotide comprises the target (dotted) and a replacement sequence (horizontal hatching on arrow) to convert the deletion in the Tet resistance gene (blank box by horizontal hatching) on the vector to a functional wild type TetR gene (horizontal hatching). In this example, the vector contains an ampR selectable marker (vertical hatching) for use in propagating the vector.

As the annealing temperature is lowered, the 3′ ends of oligomers anneal to the vector template, as illustrated in panel B. Because the 5′ end-containing oligonucleotides are annealed prior to lowering the annealing temperature, the 3′ ends from these molecules will likely anneal to the vector before the truncated oligomers anneal, resulting in the selective exclusion of truncated oligomers from the vector template. As in the previous example, the oligonucleotide is extended with T4 DNA polymerase such that extension terminates at the junction of the deletion and vector sequence (open arrow). The extension product is denatured and annealed to a universal extension primer that bridges the 5′ and 3′ ends of the extension product as shown in panel C. Extension of this extension primer generates a fully complementary extension product (illustrated in panel D) that can be converted into the ligated product illustrated in panel E by ligation, transformation of E. coli, and selection with tetracycline as described in the previous example.

This method of cloning can also be used to select and clone full-length linear cDNA target sequences from universally or randomly primed cDNA libraries. By adding an excess of vector template, different target sequences with different abundance can be cloned in an intramolecular ligation reaction without the need to digest prior to circularization.

### Example

### Insert A Cloning of Oligonucleotide Comprising a Second Replacement Sequence

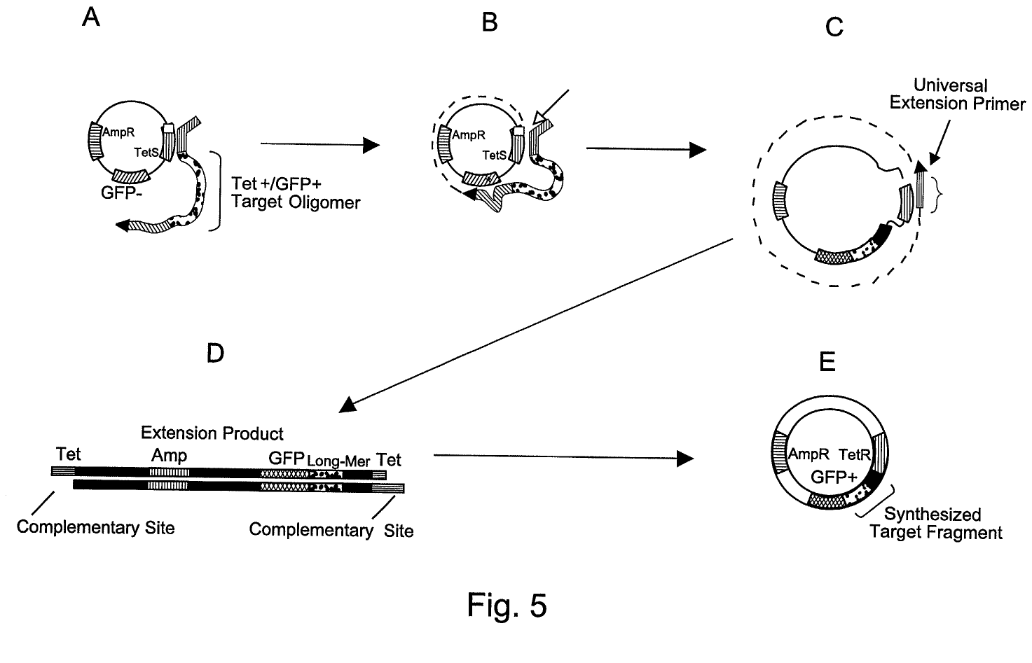

FIG. 5, Panels A-E illustrates the cloning of an oligonucleotide including the optional second replacement sequence and optional selective annealing step. Briefly, the 5′ end of an oligomer is annealed to a denatured vector (panel A). The 3′ end of the oligomer is annealed by lowering the temperature and extended with, e.g., T4 DNA polymerase. Extension terminates at the junction of the deletion and vector sequence (panel B). The resulting product is denatured, annealed and extended with an extension primer (panel C). The extension primer anneals at the junction of the tet gene and vector sequence and allows the extension of the second strand to generate a fully complementary extension product (panel D), which is ligated, transformed into E. coli, and selected with tetracycline and screened for GFP positive clones.

Thus, as depicted in panel A, an oligonucleotide containing a 5′ phosphate is annealed first at the 5′ end to a vector template. Because the 5′ end has a higher melting temperature than the 3′ end, oligomers that contain 5′ end sequences are selectively annealed to the vector, and oligomers that lack 5′ end sequences are excluded from annealing at the higher temperatures. Illustrated in panel B, as the annealing temperature is lowered, the 3′ ends of the annealed oligomers anneal to the vector template before the unannealed truncated oligomers bind, resulting in the specific exclusion of truncated oligomers from the vector template.

As in the previous example, the oligonucleotide comprises the target (dotted) and a replacement sequence (horizontal hatching on arrow) to convert the deletion in the Tet resistance gene (blank box by horizontal hatching) on the vector to a functional wild typeTetR gene (horizontal hatching). In this example, the vector contains an ampR selectable marker (vertical hatching) for use in propagating the vector.

The 3′ ends of the target oligonucleotides contain a wild type sequence for the GFP gene (forward crosshatching), and result in the conversion of the mutated vector-copy of GFP (reverse crosshatching) to a wild type GFP gene (crosshatched) upon annealing to the vector and extending, e.g., with T4 DNA polymerase such that extension terminates at the junction of the deletion and vector sequence (open arrow). The extension product is denatured and annealed to a universal extension primer that bridges the 5′ and 3′ ends of the extension product, as shown in panel C. Extending the annealed primers generates a fully complementary extension product as shown in panel D containing a GFP coding sequence fused to a target sequence. The extension product can be converted into the product illustrated in panel E, through ligation, transformation of E. coli, and selection for tetracycline resistance. Using this method, all protein encoding target sequences can be fused in-frame to GFP to allow for the screening of insertion, deletion or non-sense mutations prior to sequencing by selecting GFP positive colonies. Although GFP is used as the second nonfunctional marker in this example, many other markers known to one of skill can be employed (e.g., a selectable marker, another optically detectable marker, beta galactosidase, or the like).

Insert B

One class of embodiments (referred to here as the “insert B” embodiments) allows the incorporation of a target sequence into a cloning vector by using one or more single-stranded or double-stranded nucleic acids comprising or encoding the target as a primer. In this method, the provided vector or vector template is linear. A nonfunctional marker located on the vector is converted to a functional marker by sequences supplied by the one or more nucleic acids that comprise the target, and the vector plus insert is circularized. Selection or screening for the functional marker reduces the background of negative clones.

The vector used in the method may be single-stranded or double-stranded. The vector comprises a nonfunctional marker or a nonfunctional portion of a marker (e.g., a mutated or truncated antibiotic resistance gene). The vector or vector template as provided is linear (for example, a linear double-stranded vector may be produced by digestion of a circular double-stranded vector with a restriction enzyme, optionally an enzyme that cleaves within the nonfunctional marker). The nucleic acid insert comprises the target DNA, at least one region that is complementary to a strand of the vector (or optionally to a vector template strand, in the case of a single-stranded vector), and a replacement sequence. This replacement sequence comprises a portion of a functional version of the marker, such that integration of the replacement sequence supplied by the insert with the nonfunctional marker supplied by the vector or vector template results in a functional marker. The insert may comprise one or more single-stranded or double-stranded nucleic acids which singly or collectively comprise the target, region of complementarity to the vector or vector template, and replacement sequence.

In this method, the vector or vector template and insert are annealed. (Optionally, if the vector and/or insert is double-stranded, it may be denatured prior to the annealing step.) The nucleic acid insert is extended, preferably with an enzyme lacking strand displacement and/or 5′ to 3′ exonuclease activity (e.g., T4 DNA polymerase). The resulting product is denatured, and a primer that anneals to the 3′ end of the extended product is used in a second extension step (again, preferably with an enzyme lacking strand displacement and/or 5′ to 3′ exonuclease activity). For convenience, this primer can be designed such that it is a universal primer, which could be used in the cloning of any desired target into a particular vector by this method. Intramolecular ligation of the resulting doubly-extended product results in a circularized vector comprising a functional marker. Optionally, the doubly-extended product can be digested with one or more restriction enzymes prior to the ligation step.

The ligation can be performed in vitro, followed by transformation of cells with the circularized vector. Alternatively, ligation can occur in vivo following transformation of the doubly-extended product into cells. Either method permits screening or selection of the resulting transformed cells for cells expressing the functional marker, which cells are likely to contain a vector carrying the desired target.

In one embodiment, the nucleic acid insert is a long synthetic oligonucleotide (e.g., an oligonucleotide that is at least 100 nt, at least 150 nt, at least 200 nt, at least 250 nt, or at least 300 nt in length). Optionally, the replacement sequence is located at or near the 5′ end of the oligonucleotide. This option may obviate the need to purify the full-length oligonucleotide away from shorter oligonucleotides which lack 5′ ends as a result of failed synthesis steps prior to cloning the oligonucleotide.

Optionally, the vector or vector template can comprise a second nonfunctional marker or nonfunctional portion of a marker and the nucleic acid comprising the target can comprise a second replacement sequence, such that integration of the second replacement sequence and second nonfunctional marker results in a second functional marker. In one embodiment, the target DNA comprises an open reading frame located 5 of and in frame with the second replacement sequence, such that a fusion protein comprising the protein or peptide encoded by the open reading frame and the marker protein is expressed. This embodiment permits selection or screening of transformed cells to select or screen against some undesired clones wherein the target DNA contains e.g. an insertion or deletion that disrupts the reading frame of the marker.

The first and optional second marker can be any known to those of skill in the art, including but not limited to a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein. A nonfunctional version of such a marker may result from one or more insertion, deletion, and/or point mutation, for example.

In all embodiments, the vector can optionally comprise an additional, functional marker for use in propagating the vector.

### Example

### Insert B Cloning of Single-Stranded or Double-Stranded Nucleic Acid

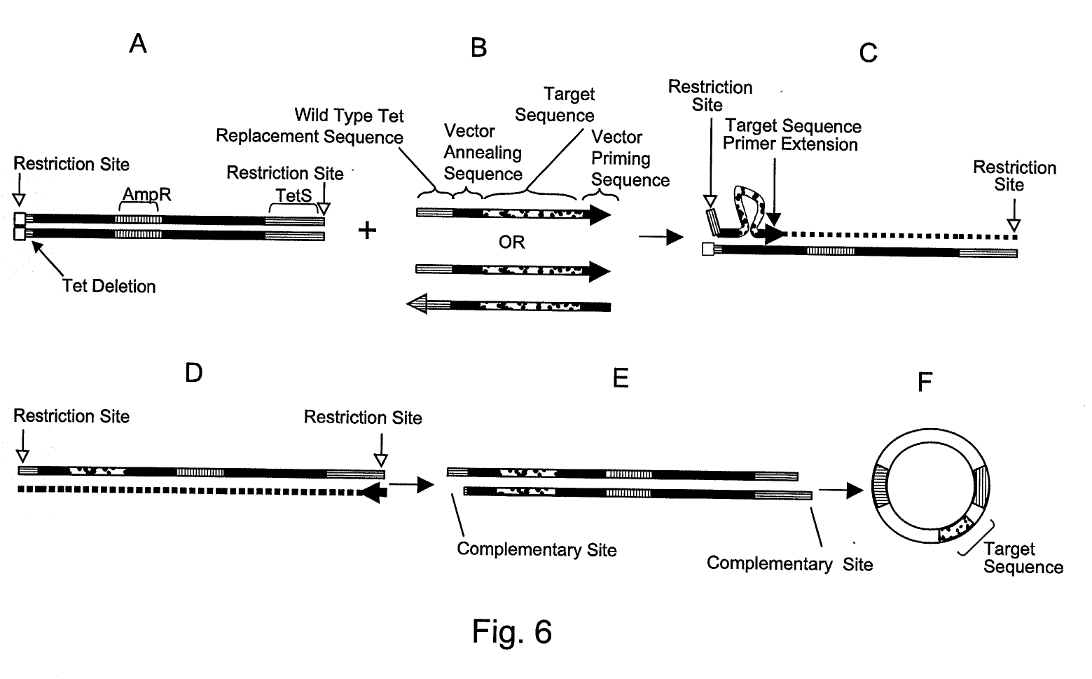

FIG. 6, Panels A-F depicts the cloning of target sequences from either single-stranded or double stranded molecules by the specific priming and extension of target sequences on a denatured linear vector template. Briefly, a double stranded vector is denatured (panel A). A single-stranded or double-stranded target sequence is annealed to the vector (panel B) and extended with T4 DNA polymerase (panel C). The resulting product is annealed with a universal reverse primer which is extended with T4 DNA polymerase (panel D). The extension product is digested (panel E) and ligated, transformed into E. coli and selected with tetracycline (panel F).

In the method depicted in FIG. 6, a mutation in a selectable marker is converted to wild type by sequences supplied by the target primers. As depicted in panel A, a vector can include two selectable markers: one marker to propagate the vector, and one mutated marker to select for clones containing the target insert. In this example, the ampicillin resistance gene (ampR) (vertical hatching) is the marker used to propagate the vector, and is shown here solely for the purpose of illustration, as any selectable marker know by those skilled in the art might be employed. The conversion of a deletion mutation in the tetracycline resistance gene (blank box by horizontal hatching) (TetS) into a wild type gene (horizontal hatching) (TetR) with sequences supplied by the insert (horizontal hatching on arrow) is used in this example as an assay to select the clones containing the target (dotted). Any selectable marker known by those skilled in the art can also be employed in this assay.

Panels A-B illustrates that any single-stranded or double stranded target DNA molecule containing regions of complementarity to a vector, referred to here as the vector annealing and priming sequences (solid filled), and replacement sequences for the mutated selectable marker can potentially be used as a primer in a cloning reaction. In this example, the double-stranded vector is denatured and annealed to the single-stranded or double-stranded target sequence. Panel C depicts an extension reaction of an annealed target primer with T4 DNA polymerase. Upon denaturation, this results in the generation of a single-stranded molecule containing target sequences that are flanked by vector sequences. T4 DNA polymerase is used in this example for the purpose of illustration, as any DNA polymerase that lacks a strand displacement activity such as Taq polymerase can be used in the extension reaction.

Panel D illustrates a second extension reaction with a universal primer to generate a double stranded fully complementary extension product containing the target sequence, the AmpR gene, and a fragmented TetR gene. Panel E depicts the complementary 5′ overhangs that result from the digestion of the extension product with a restriction endonuclease (restriction sites indicated by open arrows). Panel F depicts the final ligated product that results from the ligation, transformation into E. coli, and selection with tetracycline of positive colonies.

This method of cloning has the advantage of being able to incorporate any linear DNA sequence into a cloning vector by using the target sequence as a primer, and then joining the vector in an intramolecular reaction.

### Example

### Insert B cloning of Single-Stranded or Double-Stranded Nucleic Acid Comprising a Second Replacement Sequence

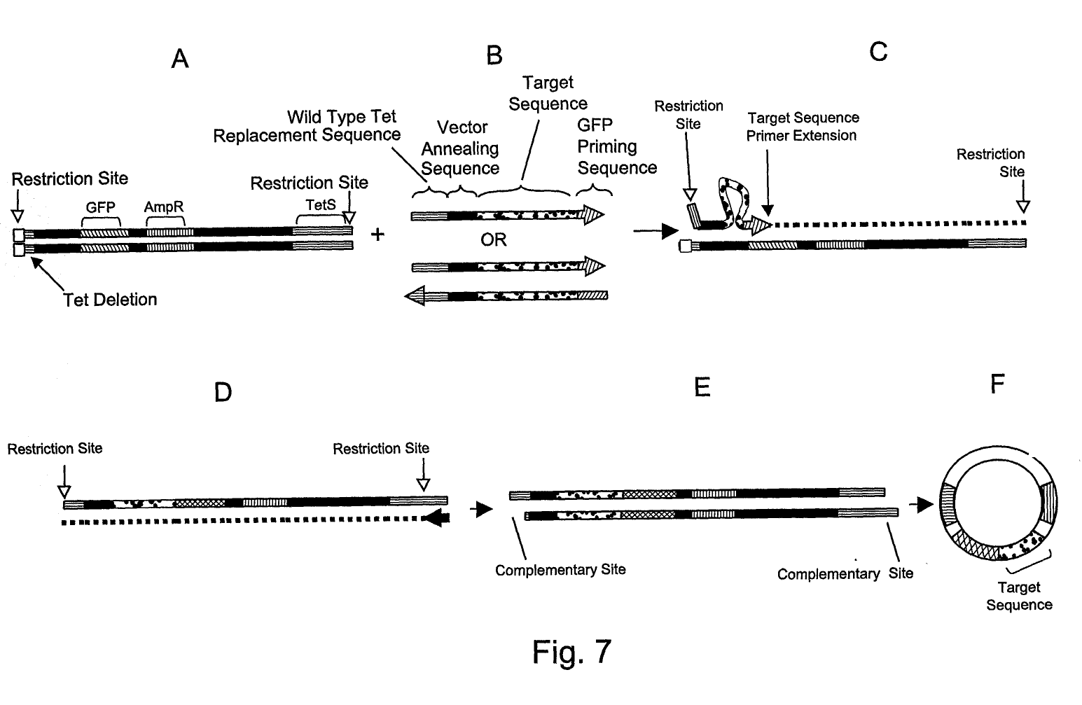

FIG. 7, panels A-F illustrates the cloning of target sequences from either single-stranded or double stranded molecules by the specific priming and extension of target sequences on a denatured linear vector template, where the nucleic acid comprising the target also comprises the optional second replacement sequence. A mutation in a selectable marker is converted to wild type by sequences supplied by the target primers. Briefly, a double stranded vector is denatured (panel A) and a single or double stranded target is annealed to the vector (panel B). The sequences are then extended, e.g., with T4 DNA polymerase (panel C). A universal primer is extended, e.g., with T4 DNA polymerase (panel D). The extension product is digested with a restriction enzyme (panel E), ligated and transformed into E. coli and selected with tetracycline to produce the product of panel F.

In the embodiment depicted in FIG. 7, as depicted in panel A, a vector can include, e.g., two selectable markers: one marker to propagate the vector, and one mutated marker to select for clones containing the target insert. In this example, the ampicillin resistance gene (ampR, vertical hatching) is the marker used to propagate the vector, and is shown here solely for the purpose of illustration, as any selectable marker know by those skilled in the art might be employed. The conversion of a deletion mutation in the tetracycline resistance gene (blank box by horizontal hatching) (TetS) into a wild type gene (horizontal hatching) (TetR) with sequences supplied by the insert (horizontal hatching on arrow) is used in this example as an assay to select the clones containing the target (dotted). Any selectable marker known by those skilled in the art can also be employed in this assay. The vector also comprises a mutated GFP (reverse cross-hatching).

Panels A-B illustrates that any single-stranded or double stranded target DNA molecule containing a region of complementarity to a vector template, referred to here as the vector annealing sequence (solid filled), and a region of complementarity to the GFP gene, referred to here as the GFP priming sequence or second replacement sequence (forward crosshatching), and replacement sequences for the mutated selectable marker can potentially be used as a primer in a cloning reaction. Panel C depicts an extension reaction of an annealed target primer with T4 DNA polymerase. Extension products containing a GFP coding sequence (crosshatched) fused to target sequences are generated. Using this method, all protein encoding target sequences can be fused in-frame to GFP to allow for the screening e.g. of insertion and deletion mutations prior to sequencing by selecting GFP positive colonies. T4 DNA polymerase is used in this example for the purpose of illustration, as any DNA polymerase that lacks a strand displacement activity such as Taq polymerase can be used in the extension reaction.

Panel D illustrates a second extension reaction with a universal primer to generate a double stranded fully complementary extension product containing the target sequence, the GFP gene, the AmpR gene, and a fragmented TetR gene. Panel E depicts the complementary 5′ overhangs that result from the digestion of the extension product with a restriction endonuclease (restriction sites indicated by open arrows). Panel F depicts the final ligated product that results from the ligation, transformation into E. coli, and selection with tetracycline of positive colonies.

Heteroduplex Cloning

One class of embodiments (referred to here as the “heteroduplex” cloning embodiments) allows the incorporation of a target sequence into a cloning vector by using one or more single-stranded or double-stranded nucleic acids comprising or encoding the target as a primer. In this method, a nonfunctional marker located on the vector is converted to a functional marker by sequences supplied by the one or more nucleic acids that comprise the target. Selection or screening for the functional marker reduces the background of negative clones. The advantage to this method is that a single universal priming and extension reaction can be used to incorporate any target sequence into a cloning or expression vector. This is achieved through the transformation of a strain of E. coli that can accept the circular hybrid molecules that contain the insert sequences of interest. One approach to isolating such a strain is to transform E. coli with a heteroduplex molecule that contains a mutated essential gene on one strand and a wild type essential gene on the other strand, and then selecting for the wild type function of the essential marker gene.

The vector used in the method can be single-stranded or double-stranded. The vector comprises a nonfunctional marker or a nonfunctional portion of a marker (e.g., a mutated or truncated antibiotic resistance gene). The nucleic acid insert comprises the target DNA, at least one region that is complementary to a strand of the vector (or optionally to a vector template strand, in the case of a single-stranded vector), and a replacement sequence. This replacement sequence comprises a portion of a functional version of the marker, such that integration of the replacement sequence supplied by the insert with the nonfunctional marker supplied by the vector or vector template results in a functional marker. The insert may comprise one or more single-stranded or double-stranded nucleic acids which singly or collectively comprise the target, region of complementarity to the vector or vector template, and replacement sequence.

In this method, the vector or vector template and insert are annealed. (Optionally, if the vector and/or insert is double-stranded, it may be denatured prior to the annealing step.) The nucleic acid insert is extended, preferably with an enzyme lacking strand displacement and/or 5 to 3′ exonuclease activity (e.g., T4 DNA polymerase). Intramolecular ligation of the resulting extended product results in a circularized heteroduplex vector comprising a functional marker.

The ligation can be performed in vitro, followed by transformation of cells capable of tolerating heteroduplexes with the circularized vector. Alternatively, ligation can occur in vivo following transformation of the extended product into such cells. Either method permits screening or selection of the resulting transformed cells for cells expressing the functional marker, which cells are likely to contain a vector carrying the desired target.

In one embodiment, the nucleic acid insert is a long synthetic oligonucleotide (e.g., an oligonucleotide that is at least 100 nt, at least 150 nt, at least 200 nt, at least 250 nt, or at least 300 nt in length). Optionally, the replacement sequence is located at or near the 5 end of the oligonucleotide. This option may obviate the need to purify the full-length oligonucleotide away from shorter oligonucleotides which lack 5′ ends as a result of failed synthesis steps prior to cloning the oligonucleotide.

Optionally, the vector or vector template can comprise a second nonfunctional marker or nonfunctional portion of a marker and the nucleic acid comprising the target can comprise a second replacement sequence, such that integration of the second replacement sequence and second nonfunctional marker results in a second functional marker. In one embodiment, the target DNA comprises an open reading frame located 5′ of and in frame with the second replacement sequence, such that a fusion protein comprising the protein or peptide encoded by the open reading frame and the marker protein is expressed. This embodiment permits selection or screening of transformed cells to select or screen against some undesired clones wherein the target DNA contains one or more insertion, deletion, or nonsense mutation that disrupts the reading frame of the marker.

The first and optional second marker can be any known to those of skill in the art, including but not limited to a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein. A nonfunctional version of such a marker may result from an insertion, deletion, or point mutation, for example.

In all embodiments, the vector can optionally comprise an additional, functional marker for use in propagating the vector.

### Example

### Heteroduplex Cloning of Single-Stranded or Double-Stranded Nucleic Acid

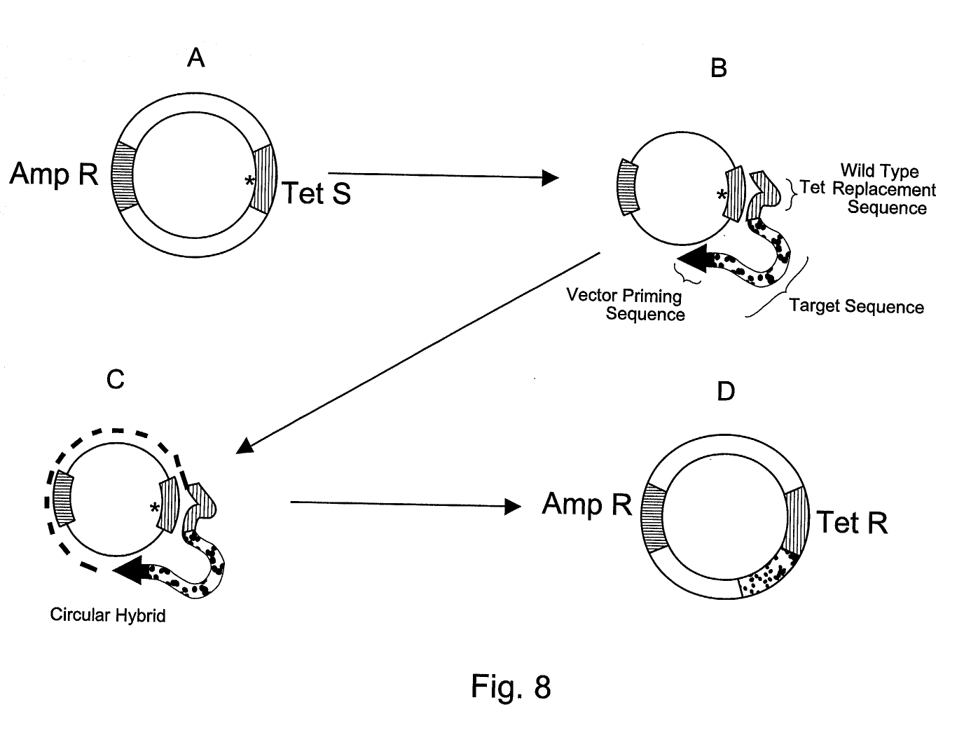

FIG. 8, Panels A-D depict the use of a linear target sequence as the sole primer in a single extension reaction to clone target sequences. In this example, a double-stranded vector comprises a selectable marker for use in propagating the vector (AmpR, vertical hatching) and a mutated, nonfunctional tetracycline resistance gene (horizontal hatching with asterisk), illustrated in panel A. The double-stranded vector is denatured and annealed to a single-stranded or double-stranded target sequence (dotted) that is flanked by a wild type replacement sequence for an essential gene at the 5′ end (horizontal hatching on arrow) and a universal vector priming sequence at the 3′ end (solid filled), as illustrated in panel B. The annealing and priming sites can be anywhere in the vector template. In this example, the selectable marker is the tetracycline resistance gene, and is used here for the purpose of demonstration. As illustrated in panel B, the annealing of a target sequence leads to replacement of the mutation in the tetracycline resistance gene with wild type sequence (horizontal hatching), allowing for the later selection of positive clones.

In panel C, the annealed target sequence primer is extended with T4 DNA polymerase, shown here for the purpose of demonstration, to generate a heteroduplex sequence. Because T4 DNA polymerase lacks strand displacement activity, the 3′ end of the extended product abuts the 5′ end of the primer. Ligation of the circular hybrid extension product and transformation of the extension reaction into a mutant strain of E. coli can result in the generation of positive clones, as illustrated in panel D. By screening for the tetracycline resistance, the 5′ ends of all target sequences can be selected.

### Example

### Heteroduplex Cloning of Oligonucleotide Comprising a Second Replacement Sequence

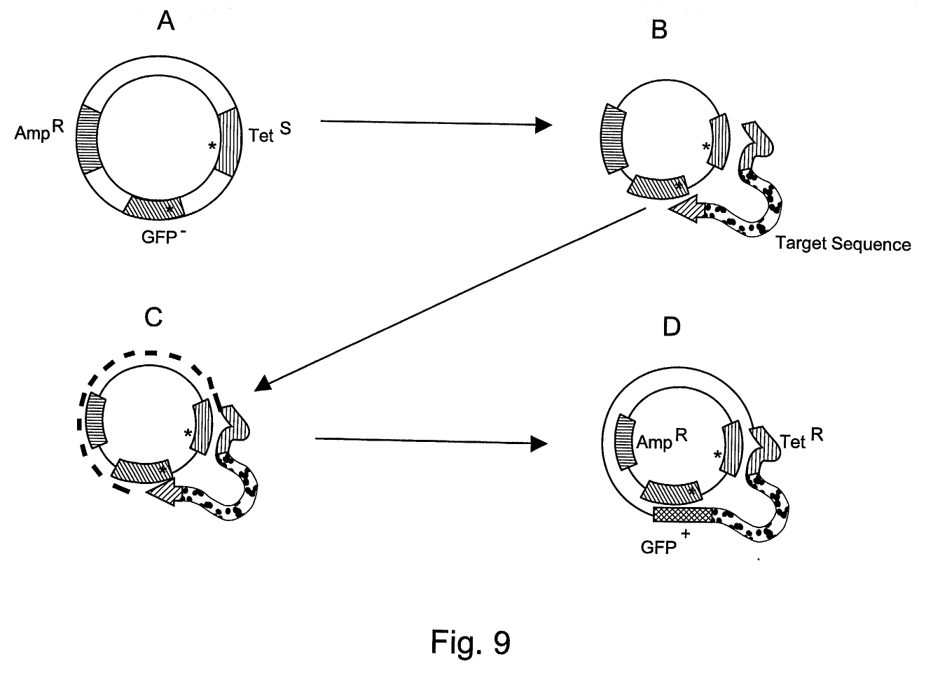

FIG. 9, Panels A-D illustrate the cloning of an oligonucleotide by the heteroduplex method. In this example, a double-stranded vector comprises a selectable marker for use in propagating the vector (AmpR, vertical hatching), a mutated, nonfunctional tetracycline resistance gene (horizontal hatching with asterisk), and a mutated GFP gene (reverse crosshatching), illustrated in panel A. The double-stranded vector is denatured and annealed to an oligonucleotide comprising a target sequence (dotted) that is flanked by a wild type replacement sequence for an essential gene mutation at the 5′ end (horizontal hatching on arrow) and a wild type replacement sequence for a GFP gene mutation at the 3′ end (forward crosshatching), as illustrated in panel B. In this example, the selectable marker is a tetracycline resistance gene, and is used here for the purpose of demonstration. As illustrated in panel B, the annealing of a target sequence leads to replacement of the mutation in the tetracycline resistance gene with wild type sequence (horizontal hatching), allowing for the later selection of positive clones. Also illustrated in panel B, the annealing of a target sequence leads to replacement of the mutation in the GFP gene with wild type sequence, allowing for the later screening of GFP positive clones. The annealing and priming sites can be anywhere in the vector template.

In panel C, the annealed target sequence primer is extended with T4 DNA polymerase to generate a heteroduplex sequence. Extension products containing a GFP coding sequence (crosshatching) fused to target oligomer sequences are generated in this step. Using this method, all protein encoding target sequences can be fused in-frame to GFP to allow for the screening of insertion, deletion, and non-sense mutations prior to sequencing by selecting GFP positive colonies. T4 DNA polymerase is used in this example for the purpose of illustration, as any DNA polymerase that lacks a strand displacement activity such as Taq polymerase can be used in the extension reaction. Because T4 DNA polymerase lacks strand displacement activity, the 3′ end of the extended product abuts the 5′ end of the primer. Ligation and transformation of the extension reaction into E. coli (e.g., in a mutS strain) can result in the generation of positive clones, as illustrated in panel D. By screening for tetracycline resistance, the 5′ ends of all target sequences can be selected.

Uses of the Invention

All of the embodiments of the present invention have significant utility in the high-throughput cloning of DNA. By decreasing the background of negative clones, the invention permits initial cloning steps to be performed in a high-throughput (e.g., 96-well, 384-well or 1536-well microtiter plate-based) format without the need to sequence many transformants, while still ensuring a very high probability that the transformants will contain the insert of interest. In cases in which a subsequent step is, for example, sequencing, the identification of positive clones can be performed by sequencing the small proportion of cases in which the clones may be negative. The high efficiency of insert capture in these embodiments also eliminates the need for normalization of vector:insert ratios, and permits cloning of very low amounts of insert DNA.

In summary, in all of these methods, employing a strategy that prevents or minimizes the regeneration of a transformation-competent molecule that lacks target sequences reduces the background. Two general ways by which this is achieved are by: 1) making the vector backbone physically incapable of supporting the transformation of bacteria and 2) designing the sequence of the vector backbone in such a way that it is incapable of supporting the transformation of bacteria. The methods for making the vector backbone physically incapable of transformation are e.g. to fragment the vector into two pieces (the “megaprimers”) which can only recombine to form a complete transformation-competent vector by joining through the interposition of the target sequence. The method for making the vector incapable of transforming bacteria via sequence design is to disrupt sequences essential either to the replication of the vector itself or to the viability of the host under selective conditions. In all of the embodiments, the insert sequences convert the vector backbone from a form incapable of supporting transformation into one competent for transforming bacteria.

A final advantage of the present invention is its use in cloning full-length genes. For instance, it is common practice to generate material for cloning by exponential amplification of small amounts of starting material by a method such as the polymerase chain reaction. There is a direct correlation between the number of rounds of amplification and the mutation frequency in the final cloned products. Reactions that incorporate extensive amplification into the cloning process are susceptible to having higher mutation rates. Hence, any cloning process, which permits cloning very small amounts of amplified material, by allowing fewer cycles of amplification to be performed, permits such amplification-induced mutations to be minimized. The present invention thereby facilitates the goal of minimizing the mutation frequency in the final cloned products in such gene-cloning efforts.

PCR Cloning

One class of embodiments (referred to here as the “PCR cloning” embodiments) allows the cloning of a long synthetic oligonucleotide that comprises or encodes the desired target into a vector by using the long oligonucleotide as a PCR primer. The long oligonucleotide (oligomer) comprises a restriction site at its 5′ end and sequence complementary to the vector at its 3′ end. By requiring the presence of the restriction site provided on the oligonucleotide, the method may obviate the need to purify the full-length oligonucleotide away from shorter oligonucleotides which lack 5′ ends as a result of failed synthesis steps prior to cloning the oligonucleotide.

The vector used in the method may be single-stranded or double-stranded. The vector can optionally comprise a selectable marker. A long synthetic oligonucleotide that is at least 100 nucleotides in length (e.g. at least 150 nt, at least 200 nt, at least 250 nt, or at least 300 nt) is provided as a first primer. The long synthetic oligonucleotide comprises the target DNA, a region that is complementary to a strand of the vector (or optionally to a vector template strand, in the case of a single-stranded vector) located 3′ of the target, and a restriction site located 5′ of the target. Preferably, the restriction site is one that is not also found in the vector. A second primer is provided which comprises a second restriction site 5′ of a region complementary to the other strand of the vector (or of the vector-vector template pair, in the case of a single-stranded vector). Again, the restriction site is preferably one that is not also found in the vector. Optionally, a third primer is provided. The optional third primer comprises a region identical to the 5′ region of the long oligonucleotide. The third primer may comprise other sequences, such as a restriction site 5′ of the region of identity to the first primer. Use of the third primer may aid in recovery of full-length product.

At least two cycles of PCR are performed to extend the provided primers. The PCR product is digested with at least one restriction enzyme. In one embodiment, the restriction sites on the first and second primers are identical and the product is digested with a single restriction enzyme.

Intramolecular ligation of the digested PCR product results in a circularized vector. The ligation can be performed in vitro, followed by transformation of cells with the circularized vector. Alternatively, ligation can occur in vivo following transformation of the digested product into cells. Optionally, after PCR amplification, the double-stranded product is digested with an enzyme that cleaves the provided vector or vector template but not the PCR product. For example, digestion with Dpn I would cleave and selectively degrade a methylated parental plasmid but not the PCR-amplified vector containing insert. This optional step may reduce background of negative clones containing only parental vector.

Optionally, the vector or vector template can comprise a nonfunctional marker or nonfunctional portion of a marker. The long oligonucleotide comprising the target can comprise a replacement sequence. The replacement sequence comprises a portion of a functional version of the marker, such that integration of the replacement sequence and nonfunctional marker results in a functional marker. In one embodiment, the target DNA comprises an open reading frame located 5′ of and in frame with the replacement sequence, such that a fusion protein comprising the protein or peptide encoded by the open reading frame and the marker protein is expressed. This embodiment permits selection or screening of transformed cells to select or screen against some undesired clones wherein the target DNA contains one or more insertion, deletion or non-sense mutation that disrupts the expression of the marker.

The optional marker can be any known to those of skill in the art, including but not limited to a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein. A nonfunctional version of such a marker may result from an insertion, deletion, or point mutation, for example.

Example advantages of various embodiments of the PCR cloning method include the following: This method can be used to specifically select the 5′ends of all oligomers. This method can be used to specifically select the 3′ends of all oligomers. This method can be used to specifically select some oligomers that lack internal deletions. This method utilizes universal annealing sequences for the cloning of all syntheses, and simplifies the production-scale cloning of all oligomers to one standard annealing condition. Oligonucleotide purification is not required. The ligation reaction is an intramolecular reaction, which can reduce mutation frequencies by allowing the cloning of a smaller amount of product using fewer PCR cycle numbers. A large number of fragments can be screened in each transformation reaction. Vector preparation is not required. The parental vector can optionally be eliminated by Dpn I digestion. Optional co-amplification of a selectable marker (e.g., the ampicillin resistance gene) might allow for the selection of low-cycle number PCR products.

This method has many potential applications, for example in synthesis of long oligos, gene synthesis, gene replacements, mutagenesis studies, defining the regulatory elements of genes, gene characterization by complementation studies, and making fusion proteins.

### Example

### PCR Cloning

Described here is a method for the direct cloning of long oligonucleotides by priming on a vector template. This method allows the selection and cloning of long oligomers that contain desired 5′ and 3′ termini by incorporating a unique restriction site at the 5′ terminus and sequence complementary to a vector template at the 3′ terminus for each oligomer. During PCR amplification, each long oligomer is incorporated into a linear product that contains both the vector sequence and the unique restriction sites at the 5′ and 3′ ends. Digestion of the 5′ and 3′ ends with the specified restriction endonuclease allows each long oligonucleotide to be cloned directly into the vector by an intra-molecular ligation reaction. Because the parental plasmid contains methylated Dpn I restriction sites, while the PCR amplified vector lacks methylation at these sites, the parental vector can be selectively degraded using Dpn I restriction endonuclease prior to transformation to reduce the vector background.

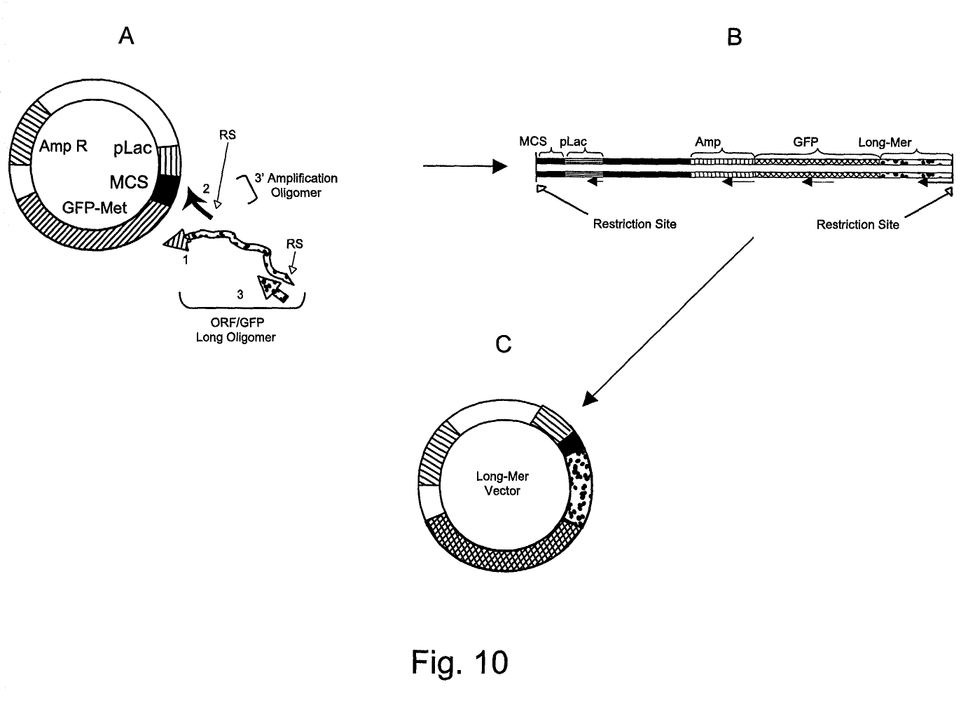

The method for cloning full-length long oligonucleotides without prior purification using long oligomers as primers in PCR amplification is illustrated in FIG. 10, Panels A-C. In the example diagrammed in panel A, the amplification of a cloning vector containing the ampicillin resistance gene (AmpR) shown in vertical hatching, a green fluorescent protein gene lacking an initiating methionine (GFP-Met) shown in forward crosshatching, a multiple cloning site (MCS, solid filled), and the lac promoter (pLac, horizontal hatching) is depicted schematically. Each PCR amplification may contain either two or three primers in the reaction. The first primer, designated 1, is a long oligomer (oligonucleotide) containing a restriction site at the 5′ terminus (RS, open arrow), a central coding sequence (dotted), and sequences complementary to the GFP gene at the 3′ terminus (replacement sequence, shown in forward crosshatching). The second primer is designated 2, and is the 3′ amplification oligomer that contains the same restriction site at the 5′ terminus as primer 1, and also contains sequences complementary to the vector. The third primer is designated 3, and contains sequences from the 5′ end of primer 1 including the RS.

In one set of reactions, primers 1 and 2 are used. During the reaction, primer 1 is directly incorporated into the PCR product without being amplified. In another set of reactions, primers 1, 2, and 3 are used. In this reaction, primer 3 is added to ensure the amplification of the 5′ terminus prior to cloning. In a test of the method on eight long oligos (287 nt), what appeared to be full-length PCR product for each reaction containing primers 1 and 2 was observed on an agarose gel. Most reactions that contained primers 1,2, and 3 also produced what appeared to be full-length product. In this test, PCR amplifications that contained three primers, primer 1 was added at 1/100 the molarity of primers 2 and 3, while in reactions containing two primers, the same number of moles of primers 1 and 2 were added.

During the amplification reaction, the long oligomer and the 3′ amplification oligomer are incorporated to generate a PCR product that is flanked by a unique restriction site as shown in panel B. The filled arrows in Panel B show the direction of transcription for each gene.

The PCR reaction is first treated with Dpn I restriction endonuclease to digest the parental vector containing methylated sites. The PCR product lacks methylated sites and is resistant to Dpn I digestion. After digesting the vector template to reduce the vector background, the specified restriction endonuclease is then added to the reaction to digest the 5′ and 3′ ends of the PCR product containing the unique site. This is then followed by an intramolecular ligation reaction to circularize the vector with the long oligomer as shown in panel C. The ligation reaction is transformed into E. coli and plated on ampicillin, and green colonies are selected to screen for transformants that lack out-of-frame mutations that result from the oligonucleotide synthesis reactions.

In a test of the method, an analysis of clones by restriction digestion and agarose gel electrophoresis showed that the reactions treated with ligase prior to transformation largely contained inserts that were larger than the vector-only fragments seen in reactions without ligase. The full-length positive fragment was approximately 1.0 kb in size, and the vector background fragment was approximately 0.7 kb in size. Twelve +Ligase samples showed inserts that were mostly larger than 0.7 kb, indicating non-vector sequences, while twelve −Ligase samples showed all clones containing a fragment approximately 0.7 kb in size, indicating vector sequence.

Each PCR product encodes a long fusion protein with a partial Lac Z protein with an initiating methionine at the N-terminus fused to a MCS open reading frame (ORF). The long oligomer ORF is fused to the MCS ORF at the N-terminus and the GFP ORF at the C-terminus. In a test of the method, sequencing results for a 287-base long oligomer confirmed the presence of the unique RS at the 5′ terminus, and showed the following mutation rate of synthesis (the EGFP with the initiating methionine was used in the results shown without screening the clones prior to sequencing): 4.2% of the clones were wild type, about 58.9% contained mutations that might have been detected by screening for GFP expression, and 78.3% of the clones contained multiple mutations. The data are summarized in Table 1.

TABLE 1
Sequence Results of PKC Genomer 2 From Long Oligomer PCR Amplifications

| | Sequencing | Wild | | | | Base |
|---|---|---|---|---|---|---|
| Genomer | Reaction | Type | Mutated | Deletions | Insertions | Changes |
| PKC2 | SPM(2)62 | | yes | 76 bp, 2 bp | | |
| PKC2 | SPM(2)63 | | yes | 40 bp, del in Sap I | | |
| PKC2 | SPM(2)66 | | yes | 43 bp, 81 bp | | |
| PKC2 | SPM(2)70 | | yes | 62 bp | | |
| PKC2 | SPM(2)75 | | yes | 123 bp | | |
| PKC2 | SPM(2)76 | | yes | 20 bp, 12 bp | | A>G |
| PKC2 | SPM(2)78 | | yes | 140 bp | | |
| PKC2 | SPM(2)80 | | yes | 1 bp, 40 bp | | |
| PKC2 | SPM(2)81 | | yes | 1 bp, 38 bp, 10 Ns | | |
| PKC2 | SPM(2)85 | | yes | 43 bp, 3 × 1 bp, 13 hp | | T>C, C>T |
| PKC2 | SPM(2)88 | | yes | >198 bp with Sap I | | |
| PKC2 | SPM(2)89 | | yes | 81 bp, 1 bp | | G>A, |
| | | | | | | C>T |
| PKC2 | SPM(2)94 | | yes | 43 bp, 81 bp, 65 bp | | |
| PKC2 | SPM(2)95 | | yes | 41 bp, 92 bp | | |
| PKC2 | SPM(2)96 | | yes | 24 bp, 1 bp, 6 bp, 20 | | |
| | | | | bp | | |
| PKC2 | SPM(2)97 | | yes | 80 bp, 20 bp | 19 bp? | |
| PKC2 | SPM(2)98 | | yes | 15 bp, 21 bp, 1 bp, 9 | | |
| | | | | bp | | |
| PKC2 | SPM(2)99 | | yes | 137 bp | | |
| PKC2 | SPM(2)100 | | yes | 38 bp, 2 × 1 bp, 2 bp | | A>G, |
| | | | | | | G>A |
| PKC2 | SPM(2)101 | | yes | >173 bp with Sap I | | A>T |
| PKC2 | SPM(2)102 | | yes | >161 bp | | A>T |
| PKC2 | SPM(2)103 | yes | | | | |
| PKC2 | SPM(2)104 | | yes | 3 bp, 6 bp, 53 bp | | |
| PKC2 | SPM(2)105 | | yes | 52 bp, 11 bp | | T>C |
| | | | | Long | Two Oligomers | |
| | | | | Oligomer | Self- | |
| | | | | Priming | Priming | |
| | | | DMF | 1/115 | 1/142 | |
| | | | PMF | 1/573.6 | 1/1418 | |
| | | | TMF | 1/90 | 1/127 | |
| | | | Genomers | 78.3% | | |
| | | | with | | | |
| | | | multiple | | | |
| | | | mutations | | | |
| | | | Genomers | ˜58.9% | | |
| | | | with | | | |
| | | | screen- | | | |
| | | | able | | | |
| | | | mutations | | | |
| | | | Overall | 4.2% | 14.7% | |
| | | | Genomer | | | |
| | | | wild type | | | |
| | | | frequency | | | |

In Table 1, “DMF” is the deletion mutation frequency, “PMF” is the point mutation frequency, and “TMF” is the total mutation frequency.

This method of oligonucleotide cloning utilizes the specific annealing and priming of all synthesized oligomers containing ORFs, and provides a novel approach to cloning full-length oligonucleotides that vary in length and quantity by selecting for the 5′ and 3′ termini. By fusing the ORFs from the long oligomers to reporter genes, a specific in vivo screening or selection method for oligomers that lack frame-shift mutations can be carried out by selecting for the presence of the marker. In this example, the EGFP (enhanced green fluorescent protein) marker is used, but could include other markers such as beta galactosidase, neomycin resistance, and tetracycline resistance. Therefore, in addition to selecting the 5′ and 3′ ends of long oligomers, this positive selection also allows for the specific isolation and recovery of full-length wild type oligomers from pools containing internal deletion products. The method involves the two assumptions, namely that the fusion proteins are functional for each unique peptide sequence synthesized and that frame-shift mutations will be identified.

Gene Assembly

One class of embodiments provides methods for assembling a double-stranded DNA of any specified sequence, beginning with synthetic oligonucleotides. (This method is herein referred to as “gene assembly” for convenience, but is not limited to the assembly of a gene-other nucleic acids of interest, e.g., genes, gene fragments, cDNAs, or the like are also conveniently assembled). In this method, oligonucleotides that are least 100 nt in length (e.g. at least 150 nt, at least 200 nt, at least 250 nt, or at least 300 nt in length) are synthesized. Each oligonucleotide comprises a subsequence of the DNA of interest. Collectively, the oligonucleotides comprise or encode the entire DNA of interest, but they need not comprise both strands or one entire single strand of the double-stranded DNA (e.g., the oligonucleotides could comprise portions of one strand and non complementary portions of the second strand of the double-stranded DNA). Optionally, the oligonucleotides are purified, by enzymatic cleavage, photocleavage, or any method known to those of skill in the art.

The oligonucleotides are then assembled to form genomers. A genomer is a DNA molecule comprising a subsequence of a larger DNA of interest (e.g., a genomer could correspond to a portion of a gene), wherein the genomer is at least 200 nucleotides (nt) (e.g., at least 300 nt, at least 400 nt, at least 500 nt, at least 600 nt, at least 700 nt, at least 800 nt) in length, and wherein one strand or portions of each strand were generated initially from synthetic oligonucleotides and thus comprise a predetermined sequence. A genomer can be single-stranded or double-stranded. Genomers can be assembled by a variety of methods. For example, a single oligonucleotide of sufficient length (e.g. at least 200 nt, 250 nt, or 300 nt) could comprise a single-stranded genomer, or a pair of complementary oligonucleotides of sufficient length could comprise a double-stranded genomer. Alternatively, a single oligonucleotide could be converted to a double-stranded genomer by any of the cloning methods provided herein (i.e., the mega primer, insert A, insert B, heteroduplex, or PCR cloning method) or other methods known to those of skill in the art. Alternatively, two or more oligonucleotides could be assembled to form a double-stranded genomer, for example by using the megaprimer, insert A, insert B, or heteroduplex methods described herein, or by using other methods known to those of skill in the art.

Optionally, at least one property of the genomers can be determined. For example, the genomers can be sequenced, their restriction enzyme digestion pattern can be checked by agarose gel electrophoresis following digestion of the genomers with at least one restriction enzyme, or transformed cells can be examined for expression of a marker protein (e.g., GFP) whose gene is fused to an ORF-containing genomer.

The genomers are assembled to form the desired full-length double-stranded DNA. Cloning methods described herein, such as the megaprimer cloning method, can be used to assemble the genomers, as can other methods known to those of skill in the art. The identity of the full-length double-stranded DNA is verified, for example by sequencing the DNA or checking its restriction enzyme digestion pattern.

### Example

### Gene Assembly

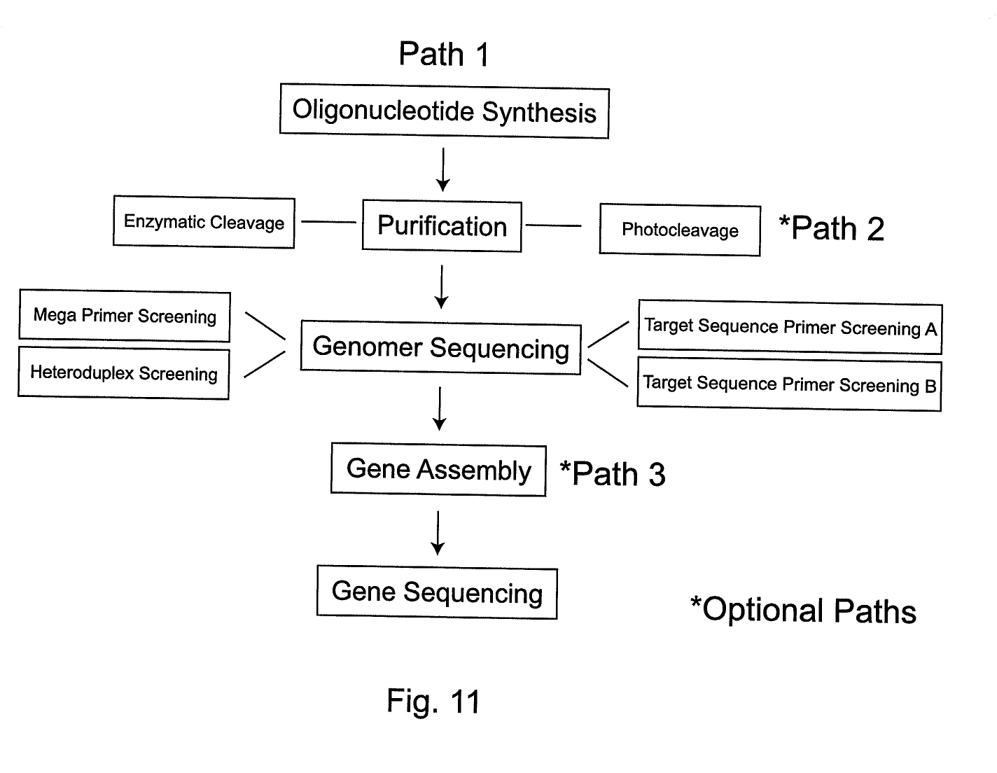

The invention includes employing unique combinations of sequential steps to generate double stranded DNA fragments of any specified sequence. The final product of this invention is referred to as a gene for the purpose of demonstration, but can include any double stranded DNA fragment of any specified sequence and of any given length that is generated by this process. Three paths are outlined here to generate synthetic gene products, and each path contains a specific set of steps that are discussed below. (See also, FIG. 11). In Path 1, six steps are specified: oligonucleotide synthesis, oligonucleotide purification (e.g., by enzymatic cleavage or photocleavage), genomer assembly (e.g., by megaprimer, insert A, insert B, or heteroduplex cloning methods), genomer sequencing, gene assembly, and gene sequencing. Path 1 includes a purification step and a “genomer”-sequencing sequencing step. Genomer is discussed in greater detail below. Path 1 is optional for the generation of any DNA fragment. In Path 2, five steps are specified: oligonucleotide synthesis, genomer assembly (e.g., by megaprimer, insert A, insert B, or heteroduplex cloning methods), genomer sequencing, gene assembly, and gene sequencing. The purification step has been omitted. Path 2 is optional for the generation of any DNA fragment. In Path 3, five steps are specified: oligonucleotide synthesis, oligonucleotide purification (e.g., by enzymatic cleavage or photocleavage), genomer assembly (e.g., by megaprimer, insert A, insert B, or heteroduplex cloning methods), gene assembly, and gene sequencing. The genomer-sequencing step has been omitted. Path 3 is optional for the generation of any DNA fragment. The steps for all paths are discussed in greater detail below. Modifications of these paths to include or omit steps, as desired, can be performed to produce a target DNA of interest.

A significant obstacle to utilizing long oligos is that the percentage of full-length material for oligos decreases significantly as a function of overall length. Moreover, the probability that an oligo will contain a mutation (such as a deletion) increases as a function of length. In order to benefit from the cost effectiveness and process robustness of using fewer, larger oligos in gene synthesis reactions, subsequent steps have been designed, and are discussed below to overcome the problems introduced by the use of such long oligos.

As the efficiency of oligonucleotide synthesis increases, the likelihood that genomer assembly will require an oligonucleotide purification step diminishes. Table 2 shows the calculated yields of full-length oligomers based on different coupling efficiencies.

TABLE 2
The Yield of Full-length Oligomers Based on the Efficiency of Synthesis

| Efficiency/ | | | | | | | | |
|---|---|---|---|---|---|---|---|---|
| Oligo Length | 100 | 200 | 300 | 400 | 500 | 600 | 700 | 800 |
| 0.999 | 91% | 82% | 74% | 67% | 61% | 55% | 50% | 45% |
| 0.995 | 61% | 37% | 22% | 14% | 8% | 5% | 3% | 2% |
| 0.990 | 37% | 14% | 5% | 2% | 1% | 0% | 0% | 0% |
| 0.985 | 22% | 5% | 1% | 0% | 0% | 0% | 0% | 0% |
| 0.980 | 14% | 2% | 0% | 0% | 0% | 0% | 0% | 0% |

For example, when the efficiency of synthesis increases from 99.5% to 99.9%, the calculated yield of 800-mer oligonucleotides increases from 2% to 45%. Therefore, by increasing the coupling efficiency of oligonucleotide synthesis, a greater proportion of the end products will be full-length, reducing the need to purify oligonucleotides. Path 2 is optional, and is based on a coupling efficiency that is increased to a point where oligonucleotide purification is no longer required.

As the mutation rate of oligonucleotide synthesis decreases, the likelihood that genomer sequencing will be required diminishes. Table 3 shows the calculated number of colonies that are required to select a clone that lacks mutations, based on the mutation rates of synthesis.

TABLE 3
The Expected Number of Colonies Required to Select WT Oligomers

| | | | | | |
|---|---|---|---|---|---|
| 1400 | 4 | 1116 | 1290410 | 1546241422 | 1920852558943 |
| 1300 | 4 | 676 | 472332 | 341114635 | 254742613306 |
| 1200 | 3 | 410 | 172889 | 75252928 | 33783852244 |
| 1100 | 3 | 248 | 63283 | 16601466 | 4480399481 |
| 1000 | 3 | 150 | 23164 | 3662431 | 594188589 |
| 900 | 2 | 91 | 8479 | 807965 | 78801027 |
| 800 | 2 | 55 | 3103 | 178244 | 10450557 |
| 700 | 2 | 33 | 1136 | 39322 | 1385948 |
| 600 | 2 | 20 | 416 | 8675 | 183804 |
| 500 | 2 | 12 | 152 | 1914 | 24376 |
| 400 | 1 | 7 | 56 | 422 | 3233 |
| 300 | 1 | 4 | 20 | 93 | 429 |
| 200 | 1 | 3 | 7 | 21 | 57 |
| 100 | 1 | 2 | 3 | 5 | 8 |
| WT | 0.9990 | 0.9950 | 0.9900 | 0.9850 | 0.9800 |
| Fre- | | | | | |
| quency | | | | | |

For example, when the efficiency of synthesis increases from 99.5% to 99.9%, the calculated number of colonies that are required to select a mutation-free clone with an 800 base pair insertion decreases from 55 to 2. Therefore, by lowering the mutation rate of synthesis, a greater proportion of clones will contain mutation-free inserts, reducing both the number of clones selected and the need to sequence genomers. Path 3 is optional, and is based on a mutation rate that is decreased to a point where genomer sequencing is no longer required.

The first step listed in FIG. 11 is the design and synthesis of oligonucleotides using standard reagents and protocols known to those skilled in the art, and is present in all paths. The specific design of the oligos (oligonucleotides) depends upon which of the embodiments described below is employed in the overall synthetic scheme (for example, an oligonucleotide could include one or more regions of complementarity with other oligonucleotides or a region complementary to a sequencing primer). This step optionally includes modifications of standard reagents and protocols as required for the generation of target DNA fragments.

The second step listed is oligonucleotide purification, and is present in paths 1 and 3. Methods for purifying full-length oligonucleotides using a site-specific nicking endonuclease are described below and other methods are available in the art (and are also discussed below). Briefly, purification by enzymatic cleavage involves two reactions. In the first reaction, target oligonucleotides are annealed to a bait oligomer that contains sequences complementary to a 5′ universal tag sequence on the target oligonucleotides, and a 3′ biotin, which can be immobilized by binding to beads coated with streptavidin. The biotin and streptavidin are illustrated solely for the purpose of demonstration, as any solid substrate that can bind to the bait oligomer and immobilize the target oligonucleotides can be used. The annealing of target oligomers to bait oligos creates an N.BstNBI recognition/cleavage site that specifies cleavage at the junction between the 3′ proximal end of the tag and the 5′ proximal end of the target sequence. In the second reaction, the N.BstNBI enzyme cleaves the immobilized and annealed tagged oligomers to generate target oligonucleotide sequences with phosphorylated 5′ ends.

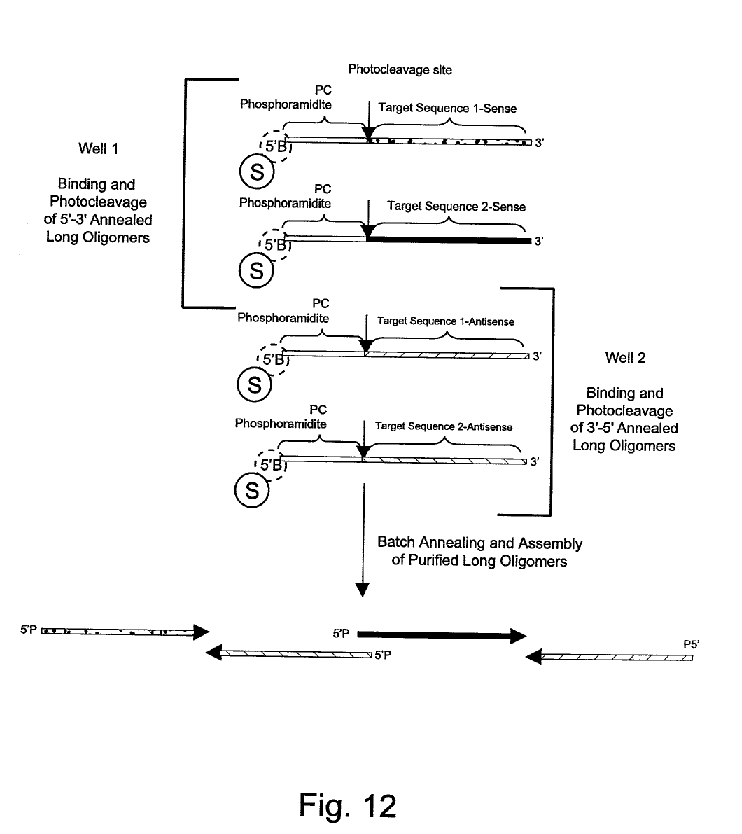

FIG. 12 illustrates a method for purifying full-length oligonucleotides using photocleavage purification, and involves two reactions. In the first reaction, target oligonucleotides (each indicated in a different pattern) containing a phosphoramidite (blank/unfilled) that has a photocleavable linkage to biotin at the 5′ end (dotted circle) are bound to beads coated with streptavidin (solid circle). In the second reaction, UV light cleaves the immobilized oligomers at the arrow to generate target oligonucleotide sequences with phosphorylated 5′ ends. These reactions are based on photocleavable reagents that are supplied by Glen Research. In this example, the purification of four different oligomers is depicted schematically. The sense target oligos can be purified by binding and cleavage in one well while the antisense target oligos could be purified by binding and cleavage in another well, followed by batch annealing and assembly as shown.

In addition to the oligonucleotide purification methods described above, any of the methods known to those skilled in the art, such as (for example) gel purification, high-performance liquid chromatography, 5′ trityl-ON purification, attachment of a removable 5′ affinity label for affinity purification, and so on can also be utilized.

The third step listed in FIG. 11 is “genomer” assembly, and is present in all paths. A genomer (a contraction of gene monomers) is any single-stranded or double stranded DNA molecule that is at least 200 nt or bp in length (e.g., at least 300, at least 400, at least 500, at least 600, at least 700, or at least 800 nt or bp). The genomer generally encodes part, rather than all of a coding nucleic acid of interest (e.g., part of a gene or cDNA). At least one strand or portions of each strand of a genomer are initially generated synthetically, and, thus, the genomer contains a predetermined sequence. The nature of a genomer is that it is a discrete subunit of a gene, and contains sequences that include but are not limited to promoter sequences, coding sequences, exon sequences, intron sequences, untranslated sequences, and enhancer sequences. Typically, genomers are of such a length (e.g., 450-800 bp) that as physical clones in a known plasmid vector, they can be fully sequenced by existing technology to deliver high quality sequencing data (data with a high PHRED score, for example) over the entire sequence length. They are also of such a length that when generated by cloning from synthetic oligonucleotides, the probability that they will contain any deviations from the intended sequence is low, typically, a probability of between about 0.05 and about 0.5. In other words, the length of genomers is typically limited either by the available length of high-quality sequence read or by the mutation frequency resulting from the generation of synthetic genomers, whichever results in a shorter genomer. Genomers can exist as monomers or can be assembled into a larger fragment. Genomers may be generated using one or more oligonucleotides, megaprimers, or by any other method known to those skilled in the art. Genomers may either be propagated by cloning, or may exist as an uncloned fragments. Additional sequences may be joined to a genomer by methods including DNA synthesis, polymerase chain reaction (PCR) amplification, primer extension, ligation, and other methods known to those skilled in the art, to permit the cloning, expression, and mutational analyses of target sequences.

Very long single-stranded oligonucleotides can be designed and synthesized for the purpose of generating genomer clones. The extreme length of these oligonucleotides results in two principal advantages, and is enabled by innovations incorporated into later steps. First, the length of these oligonucleotides (typically, from 250 nt to 800 nt in length) will be such that no more than two oligonucleotides are generally required to generate a genomer, thereby minimizing the type of undesired annealing interactions between oligonucleotides common to existing methods of gene synthesis. In other words, such long oligos ensure the robustness and standardization of high-throughput genomer assembly. Second, very long single-stranded oligonucleotides can reduce overall gene synthesis costs significantly due to two factors: 1) a dominant component of the cost of DNA synthesis is the length-independent cost of processing an oligonucleotide, and 2) longer oligonucleotides minimize the total amount of overlap required between oligonucleotides in annealing-extension schemes, since the total amount of overlap depends upon the overall number of oligonucleotides for oligomers of a specified length, as opposed to the overall length of the finished double-stranded sequence.

Genomers can be generated from one, two, or more oligonucleotides, and a variety of methods can be utilized to assemble, clone, and screen for mutation-free genomer sequences. In one embodiment, a synthetic oligonucleotide is cloned by the insert A method described above to produce a double-stranded genomer. Use of the optional second replacement sequence permits screening against a subset of insertions, deletions, point mutations or the like, prior to optional sequencing of the genomer. In another embodiment, a synthetic oligonucleotide is cloned by the insert B method described above to produce a double-stranded genomer. Use of the optional second replacement sequence also permits screening against these insertions, deletions, point mutations, etc., prior to optional sequencing of the genomer. In another embodiment, a synthetic oligonucleotide is cloned by the heteroduplex method described above to produce a double-stranded genomer. Use of the optional second replacement sequence also permits screening against the insertions, deletions, point mutations, etc., prior to optional sequencing of the genomer. In another embodiment, a synthetic oligonucleotide is cloned by the megaprimer method described above to produce a double-stranded genomer. Use of the optional replacement sequence permits screening against the insertions, deletions, point mutations, etc., prior to optional sequencing of the genomer. In yet another embodiment, a synthetic oligonucleotide is cloned by the PCR cloning method described above to produce a double-stranded genomer. Use of the optional replacement sequence permits screening against the insertions, deletions, point mutations, etc., prior to optional sequencing of the genomer. Alternatively, two or more oligonucleotides are assembled by the insert A, insert B, heteroduplex, or megaprimer cloning methods described herein. Genomers can also be assembled from one or more oligonucleotides by various methods known to those of skill in the art, for example extension of two oligonucleotides with complementary 3′ ends with Taq or T4 DNA polymerase.

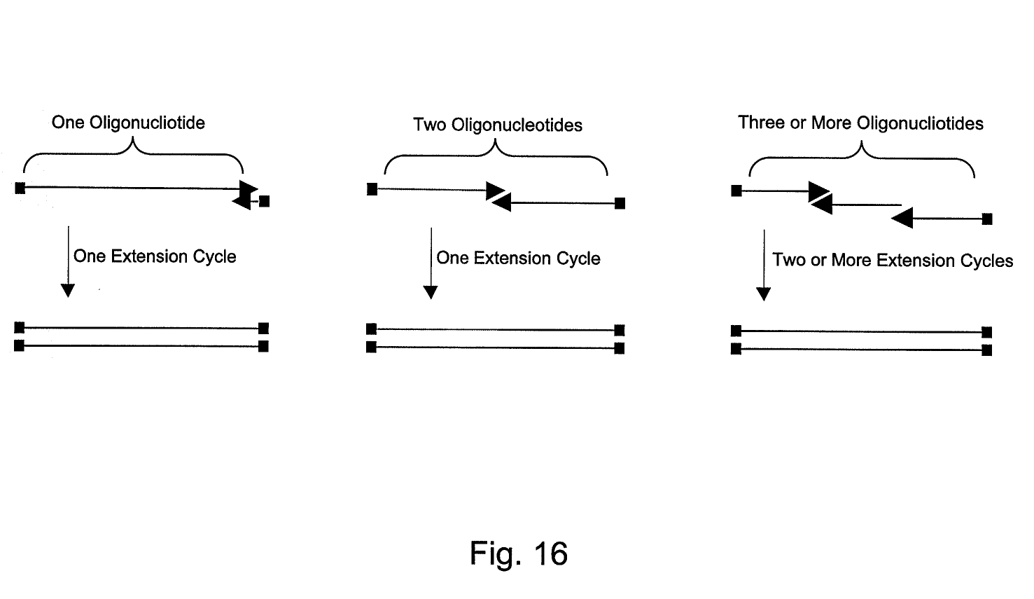

Additional details on one embodiment of genomer synthesis from oligonucleotides is found in FIG. 16. As shown, one or more rounds of polymerase-mediated extension can be used to make a genomer of interest.

The fourth step listed in FIG. 11 is genomer sequencing using standard reagents and protocols known to those skilled in the art, and is present in paths 1 and 2. This step involves performing a single-pass sequencing reaction using a universal primer to confirm genomer clones. As discussed above, this step is optional, and is based on the error rate of synthesis and the sensitivity of the screening method utilized. As the mutation rate due to synthesis is lowered, and as more mutations are detected by screening, then requirement for genomer sequencing diminishes.

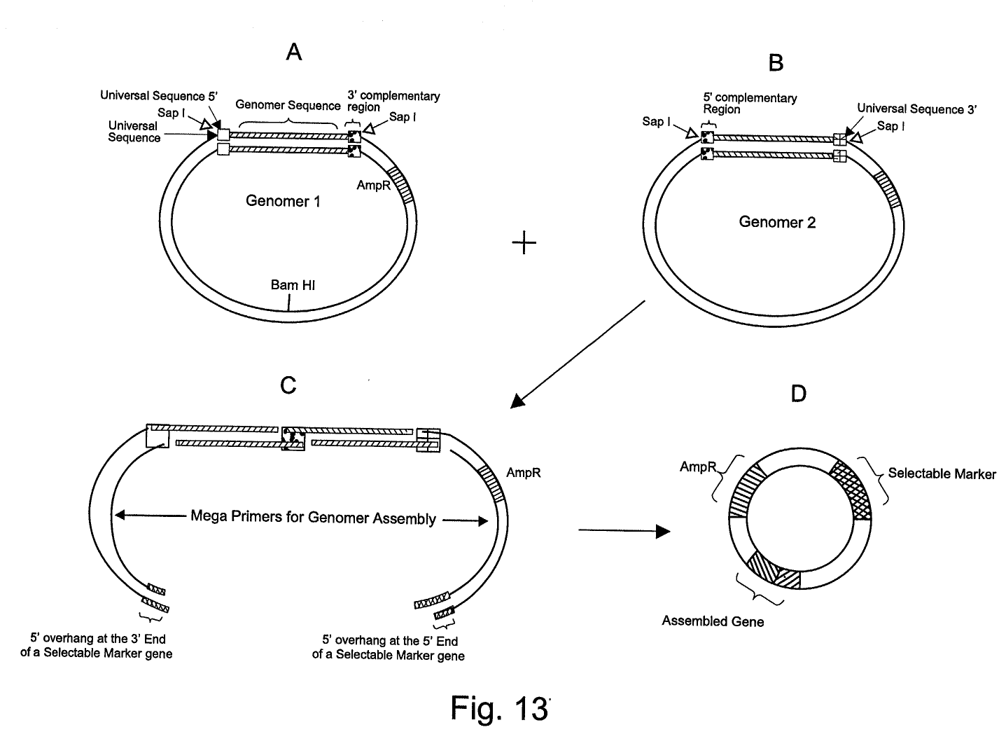

The fifth step listed in FIG. 11 is gene assembly, and is present in all paths. The assembly of genes is depicted here for the purpose of demonstration, as any full-length target sequence assembled from partial target sequences can be included in this process. Full-length target sequence is any desired double stranded DNA sequence joined from smaller partial target sequences. This step involves the assembly of genomers, as in the example illustrated in FIG. 13, panels A-D. In panels A-B, two different cloned genomers (forward and reverse crosshatching) are digested with Sap I (at open arrow) to generate linear double stranded target sequences with overlapping sequences. The Sap I restriction site flanking each genomer is illustrated for the purpose of demonstration, as any restriction enzyme recognition site that will allow cleavage to occur within the genomer may be used. The overlapping sequences within the genomer clones (dotted box) are referred to as complementary region 3′ at the 3′ end of genomer 1, and complementary region 5′ at the 5′ end of genomer 2. There are also universal sequences at the 5′ end of genomer 1 (open box) and the 3′ end of genomer 2 (dashed box). These universal sequences contain universal priming sites for primers and megaprimers used in the generation of full-length genes. Following Sap I digestion (and BarnHI digestion to cleave the vector), extension reactions are performed with T4 DNA polymerase in the presence of dATP and dTTP to generate single-stranded sites composed of dCTP and dGTP nucleotides at the ends of the genomers.

In panel C, the genomers are denatured and annealed to each other through the single-stranded complementary regions. Megaprimers are also annealed to the genomers though the universal sequences. After annealing, the primers are extended, and the extension products are digested to generate a linear vector that is joined to assembled genomers, as illustrated. The digestion reaction disrupts the selectable marker gene (crosshatched), and allows for the selection of the circularized clone. In this example, AmpR (vertical hatching) is included as an additional selectable marker.

In panel D, the digested extension products are ligated, transformed into E. coli., and selected using the regenerated selectable marker to isolate positive colonies. A selectable marker may include the origin of replication, the ampicillin resistance gene, the tetracycline resistance gene, or any other selectable marker known to those skilled in the art. Megaprimers are illustrated in this example for the purpose of demonstration, as any method that allows the cloning of genomers may be applied. These methods include versions of the heteroduplex, the insert A, and the insert B methods described above, or other methods that allow the assembly and cloning of genomers.

Oligo Synthesis, Cloning, Purification

The present invention includes the synthesis of oligonucleotides, the assembly of synthesized oligonucleotides into genomers and larger nucleic acids of interest and the cloning of oligonucleotides, genomers and oligonucleotides of interest. Cloned nucleic acids can be expressed, selected for activity and the like.

An introduction to available methods for oligonucleotide synthesis, cloning and selection is found available, e.g., in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)); PCR Protocols A Guide to Methods and Applications (Innis et al. eds) Academic Press Inc. San Diego, Calif. (1990) (Innis) and many other references.

Host cells can be transduced with nucleic acids of interest, e.g., cloned into vectors, for production of nucleic acids and expression of encoded molecules (nucleic acids or proteins of interest, markers, or the like). In addition to Berger, Sambrook and Ausubel, a variety of references, including, e.g., Freshney (1994) Culture of Animal Cells, a Manual of Basic Technique, third edition, Wiley-Liss, New York and the references cited therein, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla. provide additional details on cell culture, cloning and expression of nucleic acids in cells.

Any nucleic acid, whether corresponding to an actual nucleic acid that exists in nature (whether natural or artificial) as well as any nucleic acid that can be made to correspond to a sequence generated in a computer system can be made according to the methods of the present invention. Sources for physically existing nucleic acids include nucleic acid libraries, cell and tissue repositories, the NIH, USDA and other governmental agencies, the ATCC, zoos, nature and many others familiar to one of skill. Databases of existing nucleic acids such as Genebank™, GeneSeq™ and the NCBI can be accessed to provide the sequences of existing nucleic acids of known sequence. Other nucleic acids, e.g., corresponding to hypothetical mutations of nucleic acids of interest, or even simply to an arbitrary nucleic acid sequence of interest can be made according to the methods herein.

Oligonucleotide synthesis can be performed using chemical nucleic acid synthesis methods. For example, nucleic acids can be synthesized using commercially available nucleic acid synthesis machines which utilize standard solid-phase methods. Typically, fragments of any length up to several hundred bases can be individually synthesized, then joined (e.g., by enzymatic or chemical ligation methods, or polymerase mediated methods) to form essentially any desired continuous sequence or sequence population. Example protocols are described below for the synthesis of long oligonucleotides (e.g., over 100 bases in length). For shorter oligonucleotides, standard chemical synthesis methods can be used, e.g., the classical phosphoramidite method described by Beaucage et al., (1981) Tetrahedron Letters 22:1859-69, or the method described by Matthes et al., (1984) EMBO J. 3: 801-05., e.g., as is typically practiced in automated synthetic methods. Similarly, many nucleic acids can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.); Gorilla Genomics, Inc., and many others.

Synthetic approaches to nucleic acid generation have the advantage of easy automation. Oligonucleotide synthesis machines can easily be interfaced with a digital system that instructs which nucleic acids to be synthesized (indeed, such digital interfaces are generally part of standard oligonucleotide synthesis devices).

In one example, suited for the synthesis of long oligonucleotides, synthesis is performed on a Genemachines Polyplex® 96 well array synthesizer using the protocol 07_{—}06_{—}00_Toff20mer.pro that comes from the manufacturer. The synthesis protocol incorporates the phosphotriester method utilizing a standard terminal-Trityl off step for a 20-mer-synthesis reaction, with the following modifications: 1) A long drain step is carried out at the end of each cycle. 2) The synthesis reactions are carried out on a 50 nmol scale.

The steps in the synthesis protocol are as follows: Deblock/Hold/Deblock/Hold/Wash/Wash/Wash/Couple/Couple/Hold/Cap/Cap/Hold/Oxidize/Hold/Wash/Wash/Wash.

The post-synthesis steps are as follows: Cleave for 1 hr with NH40H; Deprotect for 12 hr@55C in NH40H.

The reagents used in the synthesis reactions are all from Glen Research, and include standard amidites, DCI as activator, 3% TCA as deblock, synthesis grade acetonitrile from Fisher, and argon from Airgas.

CPG with large pore sizes and low loads (obtained from Glen Research), were used on a low scale as a solid support for long oligonucleotide synthesis reactions.

A Method for Purifying Full-Length DNA Oligonucleotides Using Site-Specific Endonucleases

One advantage of several of the methods herein is that purification of nucleic acids is not generally required, e.g., for subsequent operations on the nucleic acids. However, purification can be performed before or after any operation, e.g., to provide a purified nucleic acid of interest. Thus, in one aspect, the present invention provides for the optional purification of any nucleic acid of interest (e.g., a long oligonucleotide, genomer, or the like), e.g., prior to use of the nucleic acid in any of the methods herein, or subsequent to production of the nucleic acid, e.g., where a purified nucleic acid is desired. Any available purification nucleic acid purification method can be used, including gel purification, chromatography, precipitation and the like. Such methods are well taught in the professional literature, e.g., in Sambrook and Ausubel, infra.

In one aspect, the present invention provides a new generally applicable method of nucleic acid purification which uses affinity binding of a target nucleic acid to an oligonucleotide, e.g., fixed to a solid support, followed by cleavage of the target nucleic acid to release the nucleic acid of interest.

Briefly, in this example purification method, purification of oligonucleotides or other nucleic acids of interest is performed by enzymatic cleavage. In a first reaction, target oligonucleotides are annealed to a bait oligomer that contains sequences complementary to a 5′ universal tag sequence on the target oligonucleotides and a 3′ biotin, which is immobilized by binding to beads coated with streptavidin. The biotin and streptavidin are illustrated solely for the purpose of demonstration, as any solid substrate that can bind to the bait oligomer and immobilize the target oligonucleotides can be used. The annealing of target oligomers to bait oligos creates a recognition/cleavage site that directs cleavage at the junction between the 3′ end of the tag and the 5′ end of the target sequence. In a second reaction, the enzyme cleaves the immobilized and annealed tagged oligomers to generate target oligonucleotide sequences with phosphorylated 5′ ends. In this example, the purification of four different oligomers is depicted schematically below.

Background

As DNA oligonucleotide synthesis proceeds, the number of active sites decreases due to a coupling efficiency that is less than 100% for each base addition. A reduction in the number of available active sites during each step in the synthesis reaction results in an overall reduction in the amount of full-length product that is synthesized. Therefore, as the length of an oligonucleotide increases, the yield of the full-length product at the end of a synthesis run decreases.

An example of the effect of oligonucleotide length on the yield of full-length product can be shown for an oligomer that is 20 bases in length, and an oligomer that is 100 bases in length. At 98% coupling efficiency, the predicted yield of a full-length 20-mer is (0.98)²⁰ or 66.8%, while the predicted yield of a full-length 100-mer is (0.98)¹⁰⁰ or 13.3%.

As the need for the synthesis of longer oligonucleotides becomes greater, purification methods can be used to allow the specific isolation and recovery of small fractions of full-length oligomers from pools containing truncated oligomers that include n-1 and n-2 termination products. This purification method uses a site-specific endonucleases to cleave at the junctions between the 3′ ends of tag sequences and the 5′ ends of target sequences to generate full-length oligomers with 5′ phosphates.

Method

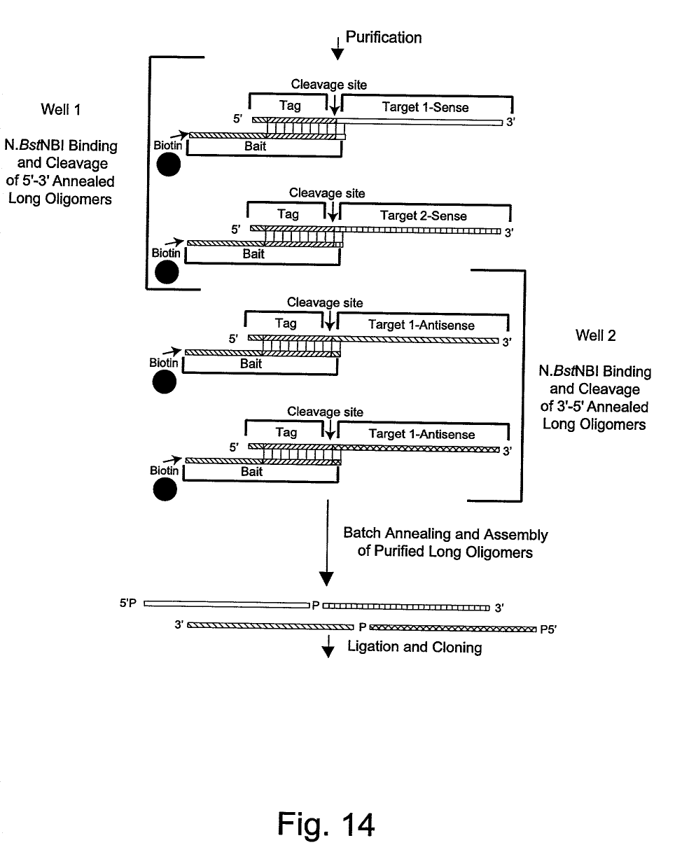

A method for purifying full-length nucleic acids of interest, such as synthetic oligonucleotides, using a site-specific nicking endonuclease is diagrammed in FIG. 14. In this example, the purification of four different oligomers using an annealing step and a cleavage reaction is depicted schematically. Two regions are defined for each synthesized oligomer. The first region is the target sequence, which contains the full-length sequence to be purified. Each target region is shown as a different pattern to indicate four different sequences. The second region is the tag sequence. The same tag sequence is present in all four oligomers. An additional pattern is used to show this 5′ tag sequence, which can vary in length. The short forward hatched section within each tag denotes a recognition/cleavage site for a nicking endonuclease, such as N.BstNBI:

5′ . . . GAGTCNNNN↓N . . . 3′

3′ . . . CTCAGNNNN N . . . 5′

The N.BstNBI enzyme recognizes GAGTC and cleaves 4 bases downstream of the recognition site denoted by an arrow (see, e.g., the New England Biolabs catalog, 2000 for a description of this enzyme). The N base that is 3′ to the cleavage site is the first base of the target sequence. Each synthesized oligonucleotide is annealed to a bait oligomer that contains sequence that is complementary to the tag sequence. The bait oligomer in this example also contains a 3′ biotin which can be immobilized by binding to beads coated with streptavidin (shown as a solid circle). Any available capture chemistry can be substituted for biotin-streptavidin (e.g., an antibody-antigen interaction). The 5′nucleotide of the bait sequence as shown is complementary to the first base of the target sequence. However, this nucleotide can be omitted and cleavage will still occur.

The reactions for the purification of oligonucleotides can be divided into two steps. In the first step, the tagged target oligomers and the bait oligomers are annealed and bound to a solid substrate. The annealing of these oligomers creates a recognition/cleavage site for the site-specific nicking endonuclease (e.g., N.BstNBI) enzyme. In the second step, the immobilized and annealed tagged target oligos are cleaved by the enzyme to generate phosphorylated 5′ ends of the target sequences.

This method of oligonucleotide purification utilizes the specific recognition and cleavage of nicking endonucleases to specifically select the 5′ end sequences of a nucleic acid of interest (e.g., any synthesized oligomer), and provides a novel approach to purifying full-length oligonucleotides that vary in length and quantity. Any endonuclease that cleaves downstream of its recognition site and that leaves either a 3′ overhang, a blunt end or a 5′ overhang with one base can be used in this application. N.BstNBI is an example enzyme that cleaves downstream of a five-base pair recognition site. (Any such enzyme can be used in the methods herein.) The 5′ nucleotide on the unnicked strand may not be required for cleavage of the target oligonucleotide by N.BstNBI, allowing the use of one universal oligomer for purifying all oligonucleotides. Should this base be essential for cleavage, four universal oligomers with four different nucleotides at this position would be sufficient for purifying any oligonucleotide that is synthesized. An additional advantage is that the bait oligonucleotide is not cleaved and can thus be reused.

In the example illustrated in FIG. 14, purified oligomers that are complementary to each other can be annealed and assembled in batch. Shown here, the sense oligomers are purified in a separate well from the antisense oligomers. The purified oligomers are released by cleaving with N.BstNBI, and then annealed. The annealed oligomers can further be ligated and sub-cloned into a vector.

The advantages of this purification method include: 1) The method can be used to specifically select the 5′ends of all oligomers; 2) This method allows the use one (or four) universal oligomers for the purification of all syntheses, and simplifies the production-scale purification of all oligomers to one standard condition; 3) The length of the bait oligo can be increased to increase specificity; 4) The cleavage step generates a 5′ phosphate that allows the ligation of target oligos without any phosphorylation reactions; and, 5) Oligonucleotides of different lengths and sequences can be purified in batch using the same universal oligo(s).

This method of purification can also be used in but is not limited to the following applications: 1) Purification of oligos for microarray construction and/or for microarray probes; 2) Synthesis of long oligos; 3) Gene synthesis; 4) Concentration of oligos; 5) Mutagenesis studies; 6) Defining the regulatory elements of genes; and 7) Gene characterization by complementation studies.

Automated Systems

In one aspect, the present invention includes automated systems that provide for the ordering of any nucleic acid of interest. In brief, an order is filled out, e.g., in a web-based order form that specifies the desired nucleic acid. This order is processed by a server that selects a method of making the nucleic acid, e.g., according to any method herein. The server then provides an automated system with instructions for the automated synthesis of the nucleic acid of interest. Thus, in one example embodiment, the system includes 1) a web based nucleic acid ordering interface; 2) system instructions that select a synthesis method; 3) apparatus for synthesizing nucleic acids or nucleic acid subsequences (e.g., oligonucleotides); 4) fluid handling components that perform any method operations herein; and 5) a QC module that tests (e.g., via sequencing or any of the other methods herein) for one or more desired property of interest.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of cloning a target DNA into a vector, the method comprising:
providing a first megaprimer;
providing a second megaprimer;
providing one or more nucleic acid that comprises or encodes the target DNA, the one or more nucleic acid comprising at least one region of complementarity to or identity with the first megaprimer and at least one region of complementarity to or identity with the second megaprimer;
extending the megaprimers; and,
intramolecularly ligating the extended product to form a functional vector.

2. The method of claim 1, wherein the one or more nucleic acid consists of one nucleic acid that at a first end comprises at least one region of complementarity to or identity with the first megaprimer and at a second end comprises at least one region of complementarity to or identity with the second megaprimer.

3. The method of claim 1, wherein the one or more nucleic acid comprises at least two nucleic acids, wherein an end of at least one of the at least two nucleic acids comprises at least one region of complementarity to or identity with the first megaprimer and an end of at least one of the at least two nucleic acids comprises at least one region of complementarity to or identity with the second megaprimer.

4. The method of claim 1, wherein the functional vector is double-stranded.

5. The method of claim 1, wherein the ligation is performed in vitro.

6. The method of claim 1, wherein the first and second megaprimers each comprise a nonfunctional marker or a fragment thereof.

7. The method of claim 6, wherein the intramolecular ligation forms a functional marker.

8. The method of claim 7, wherein the marker comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

9. The method of claim 7, comprising transforming the vector into cells and selecting or screening the cells for expression of the marker.

10. The method of claim 1, wherein either the first or the second megaprimer comprises a nonfunctional marker or a fragment thereof and the one or more nucleic acid comprises a replacement sequence comprising a portion of the marker or its reverse complement, wherein integration of the replacement sequence with the nonfunctional marker results in a functional marker.

11. The method of claim 10, wherein the nonfunctional marker comprises a mutation of a functional marker comprising at least one mutation selected from the group consisting of: a deletion, an insertion, and a point mutation.

12. The method of claim 10, wherein the functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

13. The method of claim 10, wherein the target DNA comprises an open reading frame located 5′ of and in frame with the replacement sequence.

14. The method of claim 10, comprising transforming the extended product into cells and selecting or screening the cells for expression of the marker resulting from integration.

15. The method of claim 1, wherein the one or more nucleic acid is a single-stranded DNA comprising or encoding the target DNA, the single-stranded DNA comprising at least one region identical to a region of the first megaprimer 5′ of the target DNA and at least one region complementary to the second megaprimer 3′ of the target DNA.

16. The method of claim 15, wherein the single-stranded DNA is a chemically synthesized oligonucleotide.

17. The method of claim 15, wherein extending the megaprimers comprises annealing the single-stranded DNA to the second megaprimer, extending the second megaprimer, annealing the extended second megaprimer to the first megaprimer, and extending the first megaprimer and extended second megaprimer.

18. The method of claim 17, comprising denaturing the double-stranded product formed by extending the second megaprimer prior to annealing the extended second megaprimer to the first megaprimer.

19. The method of claim 1, wherein the one or more nucleic acid is double-stranded DNA.

20. The method of claim 1, comprising digesting with at least one restriction enzyme prior to the intramolecular ligation step.

21. A method of cloning a target DNA into a vector, the method comprising:
providing a first vector or vector template comprising a nonfunctional marker or fragment thereof;
h providing one or more nucleic acid comprising or encoding the target DNA, the one or more nucleic acid comprising at least one region complementary to a strand of the first vector or vector template and a replacement sequence comprising a portion of the marker or its reverse complement, wherein integration of the replacement sequence with the nonfunctional marker results in a functional marker;
annealing the one or more nucleic acid to the first vector or vector template;
extending the one or more nucleic acid;
denaturing the resulting extended product;
providing an extension primer capable of annealing to both 5′ and 3′ ends of the extended product;
annealing the extension primer to the extended product;
extending the extension primer; and
intramolecularly ligating the doubly-extended product to form a vector comprising a functional marker.

22. The method of claim 21, wherein the first vector or vector template is a double-stranded vector, and wherein the double-stranded vector is denatured prior to annealing the one or more nucleic acid to the double-stranded vector.

23. The method of claim 21, wherein the one or more nucleic acid consists of one nucleic acid.

24. The method of claim 21, wherein the one or more nucleic acid comprises at least two nucleic acids.

25. The method of claim 21, wherein the nonfunctional marker comprises a mutation of a functional marker comprising at least one mutation selected from the group consisting of: a deletion, an insertion, and a point mutation.

26. The method of claim 21, wherein the functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

27. The method of claim 21, wherein the DNA polymerase used to extend the one or more nucleic acid or the extension primer lacks strand displacement or 5′ to 3′ exonuclease activity.

28. The method of claim 21, wherein the ligation is performed in vitro.

29. The method of claim 28, comprising transforming the ligated doubly-extended product into cells and selecting or screening the cells for expression of the marker.

30. The method of claim 21, wherein the one or more nucleic acid is a chemically synthesized oligonucleotide that is at least 100 nucleotides, at least 150 nucleotides, at least 200 nucleotides, at least 250 nucleotides, or at least 300 nucleotides in length.

31. The method of claim 30, wherein the replacement sequence is proximal to the 5′ end of the oligonucleotide.

32. The method of claim 31, wherein the 5′ end of the oligonucleotide anneals before the 3′ end.

33. The method of claim 21, wherein the first vector or vector template comprises a second nonfunctional marker or fragment thereof and the one or more nucleic acid comprises a second replacement sequence comprising a portion of the second marker or its reverse complement, wherein integration of the second replacement sequence with the second nonfunctional marker results in a second functional marker.

34. The method of claim 33, wherein the target DNA comprises an open reading frame located 5′ of and in frame with the second replacement sequence.

35. The method of claim 33, wherein the second functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein

36. The method of claim 33, comprising transforming the doubly-extended product into cells and selecting or screening the cells for expression of the second marker resulting from integration of the second replacement sequence with the second non-functional marker.

37. The method of claim 21, comprising denaturing the one or more nucleic acid prior to annealing the one or more nucleic acid to the first vector or vector template.

38. The method of claim 21, wherein the first vector or vector template comprises a functional selectable marker.

39. A method of cloning a target DNA into a vector, the method comprising:
providing a linear first vector or vector template comprising a nonfunctional marker or fragment thereof;
providing one or more nucleic acid comprising or encoding the target DNA, the one or more nucleic acid comprising at least one region complementary to a strand of the first vector or vector template and a replacement sequence comprising a portion of the marker or its reverse complement, wherein integration of the replacement sequence with the nonfunctional marker results in a functional marker;
annealing the one or more nucleic acid to the first vector or vector template;
extending the one or more nucleic acid;
denaturing the resulting extended product;
providing a primer comprising the reverse complement of the 3′ end of the extended product;
annealing the primer to the extended product;
extending the primer; and,
intramolecularly ligating the doubly-extended product to form a functional vector comprising a functional marker.

40. The method of claim 39, wherein the linear first vector or vector template is a linear double-stranded vector, and wherein the linear double-stranded vector is denatured prior to annealing the one or more nucleic acid.

41. The method of claim 40, wherein the linear double-stranded vector is produced by digestion with at least one restriction enzyme that cleaves a site located within the nonfunctional marker.

42. The method of claim 39, wherein the one or more nucleic acid consists of one nucleic acid

43. The method of claim 39, wherein the one or more nucleic acid comprises at least two nucleic acids.

44. The method of claim 39, wherein the nonfunctional marker comprises a mutation of a functional marker comprising at least one mutation selected from the group consisting of: a deletion, an insertion, and a point mutation.

45. The method of claim 39, wherein the functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

46. The method of claim 39, wherein the DNA polymerase used to extend the one or more nucleic acid or the primer lacks strand displacement or 5 to 3′ exonuclease activity.

47. The method of claim 39, wherein the ligation is performed in vitro.

48. The method of claim 47, comprising transforming the ligated doubly-extended product into cells and selecting or screening the cells for expression of the marker.

49. The method of claim 39, wherein the one or more nucleic acid is a chemically synthesized oligonucleotide that is at least 100 nucleotides, at least 150 nucleotides, at least 200 nucleotides, at least 250 nucleotides, or at least 300 nucleotides in length.

50. The method of claim 49, wherein the replacement sequence is proximal to the 5′ end of the oligonucleotide.

51. The method of claim 39, wherein the linear first vector or vector template comprises a second nonfunctional marker or fragment thereof and the one or more nucleic acid comprises a second replacement sequence comprising a portion of the second marker or its reverse complement, wherein integration of the second replacement sequence with the second nonfunctional marker results in a second functional marker.

52. The method of claim 51, wherein the target DNA comprises an open reading frame located 5′ of and in frame with the second replacement sequence.

53. The method of claim 51, wherein the second functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

54. The method of claim 51, comprising transforming the doubly-extended product into cells and selecting or screening the cells for expression of the second marker.

55. The method of claim 39, comprising denaturing the one or more nucleic acid prior to annealing the one or more nucleic acid to the first vector or vector template.

56. The method of claim 39, comprising digesting the doubly-extended product with at least one restriction enzyme prior to the intramolecular ligation.

57. The method of claim 39, wherein the first vector or vector template comprises a functional selectable marker.

58. A method of cloning a target DNA into a vector, the method comprising:
providing a first vector or vector template comprising a nonfunctional marker or fragment thereof;
providing one or more nucleic acid comprising or encoding the target DNA, the one or more nucleic acid comprising at least one region complementary to a strand of the first vector or vector template and a replacement sequence comprising a portion of the marker or its reverse complement, wherein integration of the replacement sequence with the nonfunctional marker results in a functional marker;
annealing the one or more nucleic acid to the first vector or vector template;
extending the one or more nucleic acid; and,
intramolecularly ligating the extended product to form a vector comprising a functional marker.

59. The method of claim 58, wherein the first vector or vector template is a double-stranded vector, and wherein the double-stranded vector is denatured prior to annealing the one or more nucleic acid to the double-stranded vector.

60. The method of claim 58, wherein the one or more nucleic acid consists of one nucleic acid.

61. The method of claim 58, wherein the one or more nucleic acid comprises at least two nucleic acids.

62. The method of claim 58, wherein the nonfunctional marker comprises a mutation of a functional marker comprising at least one mutation selected from the group consisting of: a deletion, an insertion, and a point mutation.

63. The method of claim 58, wherein the functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

64. The method of claim 58, wherein the DNA polymerase used to extend the one or more nucleic acid lacks strand displacement or 5′ to 3′ exonuclease activity.

65. The method of claim 58, wherein the ligation is performed in vitro.

66. The method of claim 65, comprising transforming the ligated extended product into cells capable of tolerating heteroduplexes and selecting or screening the cells for expression of the marker.

67. The method of claim 58, wherein the one or more nucleic acid is a chemically synthesized oligonucleotide that is at least 100 nucleotides, at least 150 nucleotides, at least 200 nucleotides, at least 250 nucleotides, or at least 300 nucleotides in length.

68. The method of claim 67, wherein the replacement sequence is proximal to the 5′ end of the oligonucleotide.

69. The method of claim 58, wherein the first vector or vector template comprises a second nonfunctional marker or fragment thereof and the one or more nucleic acid comprises a second replacement sequence comprising a portion of the second marker or its reverse complement, wherein integration of the second replacement sequence with the second nonfunctional marker results in a second functional marker.

70. The method of claim 69, wherein the target DNA comprises an open reading frame located 5′ of and in frame with the second replacement sequence.

71. The method of claim 69, wherein the second functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

72. The method of claim 69, comprising transforming the extended product into cells capable of tolerating heteroduplexes and selecting or screening the cells for expression of the second marker.

73. The method of claim 58, comprising denaturing the one or more nucleic acid prior to annealing the one or more nucleic acid to the first vector or vector template.

74. The method of claim 58, wherein the first vector or vector template comprises a functional selectable marker.

75. 75. A method of cloning a target DNA into a vector, the method comprising:
providing a first vector or vector template;
providing a first chemically synthesized oligonucleotide that is at least 100 nucleotides, at least 150 nucleotides, at least 200 nucleotides, at least 250 nucleotides, or at least 300 nucleotides in length that comprises or encodes the target DNA, the first oligonucleotide comprising a first restriction site 5′ of the target and a region of sequence that is complementary to a first strand of the first vector or vector template 3′ of the target;
providing a second oligonucleotide primer with a second restriction site 5′ of a region of sequence complementary to a second strand of the first vector or vector template;
performing at least one cycle of PCR amplification to extend the provided oligonucleotides;
digesting the double-stranded product with a first restriction enzyme that cleaves the first restriction site;
digesting the double-stranded product with a second restriction enzyme that cleaves the second restriction site; and
ligating the digested product.

76. The method of claim 75, wherein the first vector or vector template is a double-stranded vector.

77. The method of claim 75, wherein the first and second restriction sites are identical and the first and second restriction enzymes are identical.

78. The method of claim 75, wherein the first vector or vector template comprises a nonfunctional marker or fragment thereof and the first oligonucleotide comprises a replacement sequence comprising a portion of the marker or its reverse complement, wherein integration of the replacement sequence with the nonfunctional marker results in a functional marker, the replacement sequence located proximal to the 3′ end of the first oligonucleotide.

79. The method of claim 78, wherein the nonfunctional marker comprises a mutation of a functional marker comprising at least one mutation selected from the group consisting of: a deletion, an insertion, and a point mutation.

80. The method of claim 78, wherein the functional marker resulting from integration comprises one or more of: a selectable marker, a gene that confers cellular resistance to an antibiotic, a gene conferring resistance to ampicillin, a gene conferring resistance to tetracycline, a gene conferring resistance to kanamycin, a gene conferring resistance to neomycin, an optically detectable marker, a marker nucleic acid that encodes a green fluorescent protein, or a marker nucleic acid that encodes a beta galactosidase protein.

81. The method of claim 78, wherein the target DNA comprises an open reading frame located 5′ of and in frame with the replacement sequence.

82. The method of claim 78, comprising transforming the ligated product into cells and selecting or screening the cells for expression of the marker.

83. The method of claim 75, wherein the ligation is performed in vitro.

84. The method of claim 75, further comprising providing a third oligonucleotide primer comprising a region of sequence identical to the 5′ region of the first oligonucleotide.

85. The method of claim 75, comprising digesting the double-stranded product with an enzyme that cleaves the provided first vector or vector template but not the product of the PCR amplification.

86. The method of claim 85, wherein the enzyme is Dpn I.

87. The method of claim 75, wherein the first vector or vector template comprises a functional selectable marker.

88. A method of making a double-stranded DNA, the method comprising:
chemically synthesizing a plurality of oligonucleotides that are each at least 100 nucleotides in length and that collectively comprise a plurality of subsequences of the double stranded DNA;
assembling the plurality of oligonucleotides to form a plurality of genomers;
assembling the genomers to form the double-stranded DNA; and
determining at least one property of the double-stranded DNA.

89. The method of claim 88, wherein each of the plurality of oligonucleotides is at least 150 nucleotides, at least 200 nucleotides, at least 250 nucleotides, or at least 300 nucleotides in length.

90. The method of claim 88, wherein the genomers are double-stranded.

91. The method of claim 88, wherein the at least one property of the double-stranded DNA is determined by one or more of: sequencing the DNA, restriction enzyme digestion of the DNA, or screening for the expression of a marker fused to the DNA.

92. The method of claim 88, comprising purifying the plurality of oligonucleotides.

93. The method of claim 92, wherein the oligonucleotides are purified by enzymatic cleavage or by photocleavage.

94. The method of claim 88, comprising determining at least one property of one or more of the genomers prior to assembling the genomers to form the double-stranded DNA.

95. The method of claim 94, wherein the at least one property of the genomer is determined by one or more of: sequencing the genomer, restriction enzyme digestion of the genomer, or screening for expression of a marker fused to the genomer.

96. A method for purifying a target oligonucleotide, comprising:
providing a tagged target oligonucleotide comprising a target oligonucleotide sequence and a tag sequence 5′ of the target sequence;
providing a bait oligonucleotide comprising a region complementary to the tag;
annealing the tagged target oligonucleotide and bait oligonucleotide; and
digesting the annealed oligonucleotides with a nicking endonuclease that cleaves the tagged target oligonucleotide at a junction between the 3′ end of the tag sequence and the 5′ end of the target sequence.

97. The method of claim 96, wherein the nicking endonuclease cleaves at a site that is 3′ of its recognition sequence.

98. The method of claim 96, wherein the nicking endonuclease is N.BstNBI or N.AlwI.

99. The method of claim 96, wherein the bait oligonucleotide comprises a means for attaching the bait oligonucleotide to a solid support, and wherein the bait oligonucleotide is attached to the solid support before or after annealing the tagged target oligonucleotide and bait oligonucleotide.

100. A composition comprising: a pair of megaprimers, the pair comprising a first megaprimer and a second megaprimer, wherein each megaprimer is a single-stranded DNA molecule that comprises a distinct portion of a vector backbone and a distinct portion of an essential marker.

101. The composition of claim 100, wherein the essential marker comprises one or more of: a sequence element required for replication of a plasmid, an origin of replication, a selectable marker, a gene that confers cellular resistance to an antibiotic, a gene conferring resistance to ampicillin, a gene conferring resistance to tetracycline, a gene conferring resistance to kanamycin, or a gene conferring resistance to neomycin.

102. The composition of claim 100, wherein a first portion of the essential marker is proximal to the 5′ end of the first megaprimer and a second portion of the essential marker is proximal to the 5′ end of the second megaprimer.

103. The composition of claim 100, wherein the vector backbone comprises one or more of: an origin of replication, a selectable marker, a nonfunctional marker, an inducible promoter, or a multiple cloning site.

104. The composition of claim 100, further comprising one or more chemically synthesized oligonucleotide that comprises or encodes a target DNA.

105. A composition comprising:
a vector comprising at least one nonfunctional marker or fragment thereof, and one or more chemically synthesized oligonucleotide, wherein the oligonucleotide is at least 100, at least 150, at least 200, at least 250, or at least 300 nucleotides in length, the one or more oligonucleotide comprising at least one region complementary to at least one region of the vector and a replacement sequence, the replacement sequence comprising a portion of the marker or its reverse complement, wherein integration of the replacement sequence with the nonfunctional marker results in a functional marker.

106. A set of synthetic oligonucleotides wherein each synthetic oligonucleotide is at least 150 nucleotides, at least 200 nucleotides, at least 250 nucleotides, or at least 300 nucleotides in length, wherein the oligonucleotides collectively comprise a genomer, gene, or other full-length DNA of interest.

107. The set of synthetic oligonucleotides of claim 106, comprising at least about 2, 5, 10, 20, 48, 96, 384, or 1536 members.
